(19) United States
(12) Patent Application Publication
(10) Pub. No.: US 2023/0321412 A1
(43) Pub. Date: Oct. 12, 2023

(54) SYSTEM AND METHOD FOR MANAGING CANCER CELLS IN CSF

(71) Applicant: EnClear Therapies, Inc., Newburyport, MA (US)

(72) Inventors: Ching-Hua Tseng, Belmont, MA (US); Rajan Patel, Somerville, MA (US); Kevin Kalish, Newburyport, MA (US); Marcie Glicksman, Salem, MA (US)

(21) Appl. No.: 18/125,059

(22) Filed: Mar. 22, 2023

Related U.S. Application Data
(60) Provisional application No. 63/322,511, filed on Mar. 22, 2022.

(51) Int. Cl. A61M 27/00

(52) U.S. Cl. CPC A61M 27/006; A61M 2027/004; A61M 2202/0464; A61M 2202/20; A61M 2205/125; A61M 2205/3327; A61M 2205/3334; A61M 2205/75

(57) ABSTRACT

A method of managing cancer in a patient’s cerebrospinal fluid (“CSF”) forms a fluid circuit by fluidly communicating a set of catheters (“a catheter”) with the patient’s brain ventricle and the patient’s lumbar and, while controlling the flow rate of the CSF through the CSF fluid circuit, filters cancer cells from the CSF through the fluid circuit. The method also determines both a variable indicative of cancer cell concentration in the CSF, and at some point, that the variable has attained or passed through a threshold value (e.g., greater than a maximum threshold value or below a minimum threshold value). The method stops controlling the CSF flow rate in response to the variable attaining and/or passing through the threshold value. The CSF flow rate therefore preferably returns to a natural CSF flow rate of the patient after stopping control of the CSF flow rate. A system preferably performs these acts.

### PRIORITY

This patent application claims priority from provisional U.S. Pat. application number 63/322,511, filed Mar. 22, 2022 entitled, “RATE OF REGENERATION LIQUORPHERESIS,” and naming Ching-Hua Tseng as inventor, the disclosure of which is incorporated herein, in its entirety, by reference.

### GOVERNMENT RIGHTS

None

### FIELD

Illustrative embodiments of the invention generally relate to managing cancer cells in the body of a living being and, more particularly, various embodiments of the invention relate to managing and mitigating cancer cells within the cerebrospinal fluid of a living being.

### BACKGROUND

Cancer cells undesirably can migrate throughout the central nervous system via the cerebrospinal fluid. For example, brain metastases occur when cancer cells spread to the brain from primary tumors in other organs in the body. Metastatic tumors are common mass lesions in the brain. An estimated 24-45 percent of cancer patients in the United States have brain metastases. Moreover, approximately 200,000 new cases of brain metastases are often diagnosed in the U.S. each year. Brain metastases are the most frequent intracranial neoplasm in adults, and the most common intracranial metastatic site is the brain parenchyma. Historically, the prognosis is poor, with survival of only a few months. In the modern era, cancer-related mortality generally is associated with metastatic spread rather than the voraciousness of the primary tumor. Tumor seeding in secondary tissues therefore presents a major challenge in cancer treatment.

### SUMMARY OF VARIOUS EMBODIMENTS

In accordance with one embodiment of the invention, a method of managing cancer in a patient’s cerebrospinal fluid (“CSF”) forms a fluid circuit by fluidly communicating a set of catheters (“a catheter”) with the patient’s brain ventricle and the patient’s lumbar and, while controlling the flow rate of the CSF through the CSF fluid circuit, filters cancer cells from the CSF through the fluid circuit. The method also determines both a variable indicative of cancer cell concentration in the CSF, and at some point, that the variable has attained or passed through a threshold value (e.g., greater than a maximum threshold value or below a minimum threshold value). The method stops controlling the CSF flow rate in response to the variable attaining and/or passing through the threshold value. The CSF flow rate therefore preferably returns to a natural CSF flow rate of the patient after stopping control of the CSF flow rate. A system preferably performs the acts in the noted method.

Preferably, the method controls the CSF flow rate to be greater than the natural CSF flow rate. Moreover, the fluid circuit may have one or more additional components. Among other things, the fluid circuit may have a pump used to control the flow rate of the CSF through the CSF fluid circuit. The method therefore may stop control of the CSF flow rate by turning off the pump. The fluid circuit also may have a filter through which the CSF passes. Those in the art may select any of a number of filter types. Among other things, the filter include one or more of an electromechanical filter, a mechanical filter, a biochemical property filter, a physiochemical property filter, a temperature filter, and/or a bispecific affinity filter. The fluid circuit also may include a filter cartridge through which the CSF flows.

The variable may be a direct reading of cancer cells and/or some other value or parameter that is indicative of the concentration of cancer cells. For example, the variable may include the concentration of the cancer cells in the CSF and/or a calculated from information about the CSF. The variable may the concentration of a prescribed type of cell (e.g., red blood cells) in the CSF. It is contemplated that the variable may include an approximate or near exact concentration of cancer cells in the CSF. Among other things, the variable may be determined at least in part by using mass spectroscopy to determine the concentration of a prescribed cell type within the CSF. As another example, the variable may relate to a relationship between albumin and cancer cells in the CSF.

Some embodiments also may add a therapeutic material, such as drugs like Methotrexate to the CSF after filtering. In some embodiments, the CSF is sampled before and after filtering. In some embodiments, the CSF is sampled after at least 1 or 2 or 3 days after filtering.

Moreover, the method may determine that the variable has attained or passed through a threshold value by determining that the concentration of cancer cells in the CSF is at or below a threshold concentration value. Some embodiments maintain the CSF flow rate at a substantially constant rate. Other embodiments dynamically vary the flow rate of the CSF through the CSF fluid circuit as a function of the determined indication of cancer cell concentration in the CSF.

In accordance with another embodiment, a method of reducing cancer cells in a patient’s cerebrospinal fluid forms a fluid circuit by fluidly coupling a first catheter with the patient’s brain ventricle and fluidly coupling a second catheter with the patient’s brain lumbar and couples the first and second catheters to a pump system. Next, the method uses the pump system to increase the flow rate of the CSF flow through the CSF fluid circuit, and filters cancer cells from the CSF through the fluid circuit when the flow rate is increased. After receiving feedback relating to cancer cell concentration in the CSF (when filtering the cancer cells), the method controls the pump to enable the CSF flow rate to return to a natural CSF flow rate (in response to the received feedback).

In accordance with other embodiments, a patient cerebrospinal fluid has a plurality of catheters configured to cooperate to fluidly communicate the patient’s brain ventricle and the patient’s lumbar, and a filter in fluid communication with the plurality of catheters. The filter is configured to filter or ameliorate cancer cells from the CSF when flowing through the plurality of catheters. The system also has a spectroscopic instrument configured to produce an output signal having information relating to the CSF composition, a pump, and a flow controller operatively coupled with the pump and configured to cause the pump to control the flow rate of the CSF (e.g., greater than the natural CSF flow rate) through the plurality of catheters. The flow controller also is configured to cause the pump to permit restoral of the natural CSF flow rate in response to a determination that a variable indicative of cancer cell concentration in the CSF has attained or passed through a threshold value (e.g., see other embodiments discussed above). The variable indicative of cancer cell concentration in the CSF is a function of the output of the spectroscopic instrument.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically shows a cerebrospinal fluid circuit that may be used with illustrative embodiments of the invention.

FIG. 1B schematically shows an external catheter configured in accordance with illustrative embodiments.

FIG. 1C shows a high-level surgical flow process in accordance with illustrative embodiments of the invention.

FIG. 2 schematically lists rates of regeneration of CSF components of varying size when removed from the CSF.

FIG. 3 schematically shows how three example CSF components, TDP-43, tau, and albumin undergo periodic filtration reduction (by 50%), but achieve different steady-state levels depending on their regeneration rates.

FIG. 4A schematically shows a flow control valve circuit that may be used with illustrative embodiments.

FIG. 4B schematically shows a syringe pump dosing circuit with a drug introduced directly into the fluid line configured and usable with illustrative embodiments.

FIG. 5 schematically shows predictions of how three classes of CSF components respond to the rate of regeneration filtration.

FIGS. 6A-B schematically show a fluid path of CSF circulation past a filtration element in a) ventriculo-lumbar configuration, and b) dual lumen lumbar configuration.

FIGS. 7 and 8 schematically show two different user interfaces in accordance with illustrative embodiments.

FIG. 9 shows a process of localizing drug delivery to a target area of the brain in accordance with illustrative embodiments.

FIG. 10 schematically shows a CSF treatment system with treated and untreated fluid reservoirs.

FIG. 11 shows the flow chart of cancer filtration process

FIG. 12 shows the flow chart of a repetition cycle of filtration process

FIGS. 13A and 13B schematically show bidirectional pump circuits that enable flow in two opposite directions (FIG. 13B between right and left ventricles in the brain) in accordance with illustrative embodiments.

FIG. 14 schematically shows another system interface in accordance with illustrative embodiments.

FIG. 15 shows a block diagram of CSF maintenance system in accordance with illustrative embodiments.

### DESCRIPTION OF ILLUSTRATIVE EMBODIMENTS

In illustrative embodiments, circulation and/or treatment of a patient’s cerebrospinal fluid (“CSF”) is controlled as a function of a parameter of the CSF (e.g., the amount, concentration, presence, or absence of a target component, such as cancer cells, toxic proteins, red blood cells, virus, bacteria, or other component; viscosity of the CSF, amount of CSF, etc.). To that end, the system may include a fluid circuit with catheters respectively coupled to two CSF access sites, such as to the patient’s lumbar and brain ventricle or to the patient’s left and right brain ventricles, a flow controller configured to control CSF fluid flow rate through the fluid circuit, and one or more filters configured to filter, mitigate, and/or ameliorate the target component and/or other parameters of the CSF passing through the fluid circuit. Some embodiments also may include a mechanism to introduce a treatment component (e.g., a chemotherapy drug) into the CSF passing through the fluid circuit, such as via one of the catheters, via the flow controller, or via the filter.

After determining that a predetermined criterion has been met (e.g., a variable indicative of target component amount or concentration in the CSF has attained or passed through some static or dynamic threshold value, treatment has been performed for a predetermined amount of time, treatment has been performed for a predetermined number of CSF cycles, etc.), the system stops or otherwise adjusts controlling CSF flow rate and/or treatment, e.g., returning the CSF to its natural flow rate. Various embodiments may include one or more detectors to detect parameter(s) associated with the CSF passing through the fluid circuit, such as for determining the predetermined criterion. It should be noted that the predetermined criterion could be based on multiple parameters and need not be directly associated with the target component being treated, e.g., treatment of cancer cells could be performed until red blood cell count reaches a predetermined level such that the filter could filter cancer cells and the detector could detect red blood cells. Details of illustrative embodiments are discussed below.

It would be greatly beneficial to enhance the clearance of cancer cells from the CSF in a controlled and species-specific manner, while leaving critical components, such as albumin and red blood cells within tolerable ranges post filtration. The instant disclosure provides methods and systems for enhancing the clearance of the brain parenchyma of undesired entities, such as cancer cells and/or toxic proteins by a method and means of tailoring the removal rate to the recovery rate of various biological components in the CSF.

In some embodiments, the CSF management system removes circulating tumor cells (CTC) and/or reduces the number of circulating tumor cells from the CSF to reduce or prevent the risk of tumor spread which causes metastases in the brain. In some embodiments, the CSF management system provides for sampling the CSF at different time points and the total CSF of the patient so as to determine the number of CTCs in the CSF to facilitate the reduction of sampling error.

The removal of cancer cells and/or conditioning of specific compounds in the CSF can be tailored to the pathology of the specific disease. According to the condition the patient has or is at risk of developing, the method identifies a target bodily fluid component, such as CSF for removal of cancer cells and/or toxic proteins, such as Tau or Abeta aggregates. Based on the likely regeneration time of the target component, a filtration method and repetition time, duty cycle, and fluid flow rate are selected operable to reduce the equilibrium levels of the target component under the treatment method. Prior to initiating treatment, criteria for ending treatment are decided upon. The filtration method is operable to remove, retard, alter, deactivate, degrade, digest, immobilize, sequester, isomerize, post-translationally modify, or otherwise transform the bodily fluid component in the CSF, such as cancer cells and/or toxic proteins, such as Tau, Abeta and TDP-43.

The treatment can be applied as a preventative to patients having solid tumors outside of the brain, but not yet diagnosed in the brain, or had, or will have, a procedure whereby tumor cells may be shed into the blood or CSF compartments, or a genetic, clinical history, or environmental risk of developing neurodegenerative disease.

The treatment can be episodic with distinct treatment repetition times and duty cycles as well as a controlled fluid flow. In some embodiments, the treatment is continuous with a controlled fluid flow. The method entails identifying a potential patient for liquorpheresis treatment. The removal or conditioning of the CSF component may be targeted and specific, or more general, targeting cancer cells or more than one compound or family of compounds or a diverse group of compounds (e.g., one or more species of Abeta and tau or one or more of its hyperphosphorylated forms to treat Alzheimer’s disease) thought to be involved with the pathophysiology of a particular disease. In some embodiments, the instantly disclosed systems and methods also provide for biotherapeutic delivery of drugs, such as Methotrexate to the brain parenchyma.

The concept of ex-vivo immunotherapy using the CSF is one of the novel features of the invention whereby the CSF is filtered, such as to remove the cancer cells and then therapeutic drug, such as Methotrexate is infused into the CSF so as treat or manage cancer or prevent the proliferation of cancer. A number of conditions affecting the nervous system are believed to have a disruption in the neuroimmune axis or weak points in the blood brain barrier allowing B-cells, T-cells and the humoral and cell-mediated immune responses or the overproduction or underutilized clearance of toxic substances made within the brain and/or spinal cord. In each instance, the normal neuronal and glial architecture is victim to a broad range of toxic substances, such as cancer cells, neuro- inflammatory components and reactive oxidative stress proteins. Illustrative embodiments allow for targeted removal of cancer cells and/ or toxic proteins, such as Tau, Abeta, TDP-43 and oxidative stress proteins.

With regard to immunotherapy, present day active and passive immunotherapy treatments carry significant risk of vasogenic edema, encephalitis due to infection or generalized neuronal inflammation as well as other adverse events. By harnessing the immunotherapy components in an immobilized immunoaffinity approach, one can bring the CSF to the antibody and prevent any risk of mounting a generalized immune response against oneself or any other adverse event as the therapeutic remains within the device and does not enter the body to any significant extent. Furthermore, this eliminates or at least reduces the risk of a number of potential safety issues, including the generation of autoantibodies against systemically delivered immunotherapies, which could have devastating effects and high mortality in a subset of patients. Certain embodiments employ a cartridge- based schema that would allow for further rapid replacement of the conditioning approach. This approach can also minimize the number of interactions where the subject needs to return for follow-up care and/or additional therapeutic intervention.

Removal or amelioration of cancer cells or other biological material can be targeted based on one or more separation processes selected from the group consisting of:
i) electromechanical methods including, but not limited to: radiofrequency, electromagnetic, ultraviolet radiation, acoustic wave, piezoelectric, electrostatic, nano-, molecular/biologic force, atomic force, and ultrasonic filtration and ultrafiltration based methods,
ii) biochemical/physicochemical properties and/or temperature methods including, but not limited to: size-exclusion, pore flow, solution diffusion, protein size or secondary, tertiary or quaternary structure, diffusion, hydrophobic/hydrophilic, anionic/cationic, high/low binding affinity, chelator, magnetic or nanoparticle-based systems, and various neurochemical filtration systems,
iii) bi-specific affinity methods including, but not limited to, specific antibodies, immunotherapy-based, immuno-modulatory, ex-vivo immunotherapy using immobilized antibodies or antibody fragments, nucleic acids, receptors, anti-bacterial, anti-viral, anti-DNA/RNA, protein/amino acid, carbohydrate, enzymes, isomerases, compounds with high-low bi-specific binding affinity-based systems.
iv) flow cytometry, which facilitates rapid analysis of single cells or particles as they flow past single or multiple lasers while suspended in a solution. Each single cell is analyzed for visible light scatter and one or multiple fluorescence parameters. Lopresti et al. discloses a sensitive screening method for detecting the presence of circulating tumor cells (CTCs) using flow cytometry. Such detection methods can be utilized int the CSF management system to quantity the number of CTCs in the CSF. (See Alexia Lopresti, Sensitive and easy screening for circulating tumor cells by flow cytometry, Volume 4, Issue 14 on Jul. 25, 2019 JCI Insight. 2019;4(14):e128180).

In some embodiments, the CSF management system provides for the detecting the presence of heterogeneity in cancer cells and which subset of cancer cells are responding well to a therapeutic drug being administered. The CSF management system thus provides a real time profile of the nature of the cancer cells which allows one to determine which drug or drug combinations would be effective in treating the circulating cancer cells. In some embodiments, the CSF management system comprises a detection means, such as flow cytometry that is capable of detecting and characterizing the types of circulating tumor cells. (See Muchlinksa et al., Detection and Characterization of Circulating Tumor Cells Using Imaging Flow Cytometry-A Perspective Study. Cancers 2022, 14, 4178).

In some embodiments, the CSF is sampled before and after filtering. In some embodiments, the CSF is sampled after at least 1 or 2 or 3 days after filtering. The difference in the amount or concentration of cancer cells and/or toxic proteins in the CSF before and after filtration provides information on a threshold value. The difference in the amount or concentration of cancer cells and/or toxic proteins in the CSF immediately after filtration and at least 1 day post filtration provides information on the rate of regeneration of cancer cells and/or toxic components in the CSF.

For instance, if the cancer cells are found in the CSF after four days post filtration but not after three days post filtration then the CSF filtration process can be repeated after three days. Likewise, if the cancer cells are found in the CSF after 7 days post filtration and not after six days post filtration, then the CSF filtration process can be performed once a week. Thus, the method and accompanying CSF management system provides a way for actively monitoring the efficacy of filtration process of removing the cancer cells from the CSF and provides a way to set the repetition cycle of how often one needs undergo the CSF filtration to reduce the level of cancer cells and thereby lower the risk of brain metastasis. One can thus formulate a filtering plan as a function (linear regression, exponential regression, asymptotic function) of the difference between the first, second and third CSF sample collected during the process, where the first sample is CSF collected before filtration, second sample is CSF collected at least 60 minutes post filtration and third sample is CSF collected at least one day after filtration.

In some embodiments, the CSF management system enables the filtration of at least 5% of total CSF in the patient’s body. In some embodiments, the CSF management system enables the filtration of at least 10% of total CSF in the patient’s body. In some embodiments, the CSF management system enables the filtration of at least 20% of total CSF in the patient’s body. In some embodiments, the CSF management system enables the filtration of at least 30% of total CSF in the patient’s body. In some embodiments, the CSF management system enables the filtration of at least 40% of total CSF in the patient’s body. In some embodiments, the CSF management system enables the filtration of at least 50% of total CSF in the patient’s body. In some embodiments, the CSF management system enables the filtration of at least 60% of total CSF in the patient’s body. In some embodiments, the CSF management system enables the filtration of at least 70% of total CSF in the patient’s body. In some embodiments, the CSF management system enables the filtration of at least 80% of total CSF in the patient’s body. In some embodiments, the CSF management system enables the filtration of at least 90% of total CSF in the patient’s body. In some embodiments, the CSF management system enables the filtration of at least 100% of total CSF in the patient’s body. In some embodiments, the CSF management system provides for sampling of total CSF (100% of all CSF) in the patient’s body which allows for the determination of exact concentration of total circulating cancer cells in the patient’s CSF as a whole. Various embodiments can range between any of the above noted amounts of total CSF (e.g., 20% to 60% or the like).

An MRI of the patient’s brain and spinal cord will provide the total volume of CSF present in the body. The CSF management system keeps track of amount of CSF flowing through the system and can readily determine how much CSF has been filtered through the system. In some embodiments, the system has filtered the entire CSF volume of the patient at least once. In some embodiments, the system has filtered the entire CSF volume of the patient at least twice, thrice or four times over or five times over. In some embodiments, the system has filtered the entire CSF volume of the patient fractionally, at least 11/2 times over, at least 21/2 times, at least 31/2 times over etc. Thus, the CSF management system enables one to determine accurate concentrations of CTC in the patient since it allow for detection of CTCs over the entire volume of CSF which is unlike other systems which sample the CSF once at a discrete time point and extrapolate the value which is often plagued by sampling bias. Such sampling bias does not occur in the CSF management system due to its ability to sample the whole CSF volume of the patient.

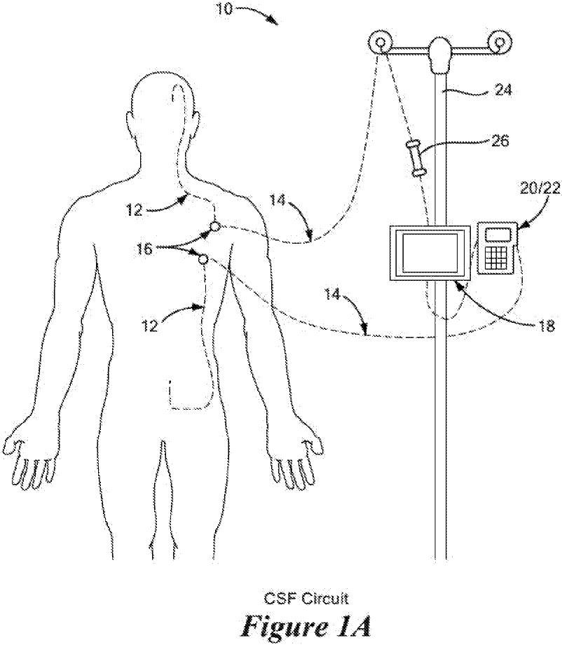

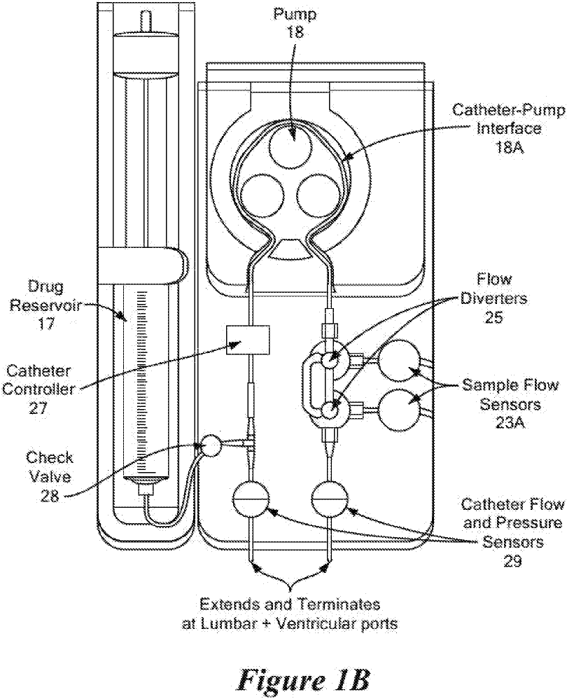

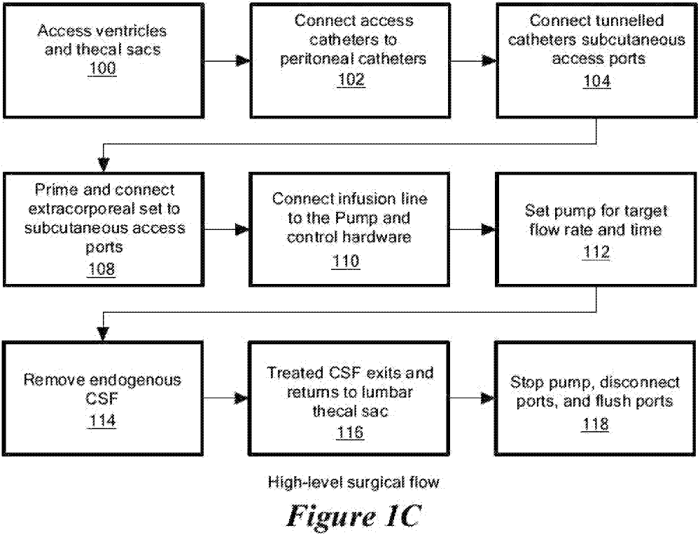

To control CSF flow within the body (e.g., through the ventricle), illustrative embodiments form a CSF circuit/channel (identified by reference number “10”) that manages fluid flow in a closed loop. FIG. 1A, for example, shows one embodiment of such a CSF circuit 10. In this example, internal catheters 12 positioned in-vivo/interior to the body fluidly couple together via the subarachnoid space. To that end, a first internal catheter 12 fluidly couples a prescribed region of the brain (e.g., the ventricle) to a first port 16, which itself is configured and positioned to be accessible by external components. In a corresponding manner, a second catheter couples the lumbar region to the subarachnoid space with a second port 16 that, like the first port 16, also is configured to be positioned and accessible by external components. The first and second ports 16 may be those conventionally used for such purposes, such as a valved Luer-lock or removable needle. The first and second internal catheters 12 thus may be considered to form a fluid channel extending from the first port 16, to the ventricle, down the spine/subarachnoid space to the lumbar, and then to the second port 16. These internal components, which may be referred to as “internal CSF circuit components,” are typically surgically implanted by skilled professionals in a hospital setting.

The CSF circuit 10 also has external components (referred to as “external CSF circuit components). To that end, the external CSF circuit components include at least two fluid conduits 14. Specifically, the external CSF circuit components include a first external fluid conduit 14, that couples with the first port 16 for access to the ventricle. The other end of the first external conduit 14 is coupled with a management system 19, which includes one or more CSF pumps (all pumps are generically identified in the figures as reference number “18”), one or more user interface/displays 20, one or more drug pumps 18, and a control system/controller 22. The fluid external fluid conduit 14 may be implemented as a catheter and thus, that term may be used interchangeably with the term “conduit” and be identified by the same reference number 14.

Illustratively, this management system 19 is supported by a conventional support structure (e.g., a hospital pole 24 in FIG. 1A). To close the CSF circuit 10, a second external catheter 14 extends from that same CSF management system 19 and couples with the second port 16 and the management system 19. This management system 19 and external catheters 14 therefore form the exterior part of a closed CSF circuit 10 for circulating the CSF and therapeutic material.

It should be noted that the CSF circuit 10 may have one or more components between the first and second ports 16 and the respective removable connections of the first and second external catheters 14. For example, the first port 16 may have an adapter that couples with the first external catheter 14, or another catheter with a flow sensor may couple between such external catheter 14 and port 16. As such, this still may be considered a removable connection, albeit an indirect fluid connection. There may be corresponding arrangements with the other end of the first external catheter 14, as well as corresponding ends of the second external catheter 14. Accordingly, the connection can be a direct connection or an indirect connection.

The first and second external catheters 12 and 14 preferably are configured to have removable connections/couplings with the management system 19, as well as their respective ports 16. Examples of removable couplings may include a screw-on fit, an interference fit, a snap-fit, or other known removable couplings known in the art. Accordingly, a removable coupling or removable connection does not necessarily require that one forcibly break, cut, or otherwise permanently break the ports 16 for such a connection or disconnection. Some embodiments, however, may enable a disconnection form the first and/or second ports 16 via breaking or otherwise, but the first and/or second ports 16 should remain in-tact to receive another external catheter 14 (e.g., at the end of life of the removed external catheter 14).

FIG. 1B schematically shows more details of the first and/or second external conduits/catheters 14. This figure shows an example of an external catheter 14 operating with other parts of the system. As shown, the system receives a drug reservoir 17 (e.g., a single-use syringe) configured to deliver a dose of therapeutic material (e.g., a drug) that fluidly couples with the catheter 14 via a check valve 28 and T-port 19 on the catheter 14. In addition, the catheter 14 is coupled with a mechanical pump 18 and also preferably includes a sample port 23 with flow diverters 25 for diverting flow toward or away from a sample port 23. The sample port 23 preferably has sample port flow sensors 23A to track samples. Some embodiments may be implemented as a simple catheter having a body forming a fluid-flow bore with removably couplable ends (or only one removably couplable end).

Illustrative embodiments show an intelligent flow system. For example, either one or both of the external catheters 14 can have a processor, ASIC, memory, EEPROM (discussed below), FPGAs, RFID, NFC, or other logic (generally identified as reference number “27”) configured to collect, manage, control the device, and store information for the purposes of security, patient monitoring, catheter usage, or communicating with the management system 19 to actively control fluid dynamics of the CSF circuit 10.

Among other things, the management system 19 may be configured to coordinate with an EEPROM 27 to control CSF fluid flow as a function of the therapeutic material infusion flow added to the CSF circuit 10 (discussed below) via the check valve 28 at the output of the drug reservoir 17. The management system 19 may be configured to coordinate with an EEPROM 27 to control CSF fluid flow as a function of the concentration of cancer cells or toxic proteins being filtered by the system. The management system 19 may speed up or slow down the pump 18 thereby increasing or decreasing the flow rate or stopping thereby allowing the flow rate to return back to natural flow rate of the CSF. The amount or concentration of cancer cells is monitored before and after filtration of CSF to generate a threshold valve. The management system 19 may stop the pump 19 thereby stopping the flow of CSF when it receives feedback that the amount or concentration of cancer cells in the CSF filtrate has reached or passed a threshold value.

As shown in FIG. 1B, one embodiment of the external catheter 14 has the noted electrically erasable programmable read-only memory, EEPROM 27, (or other logic/electronics) that can be implemented to accomplish a variety of functions. Among others, the EEPROM 27 can ensure that the CSF circuit 10 and its operation is customized/individualized to a patient, a treatment type, a specific disease, and/or a therapeutic material. For example, in response to reading information stored in the EEPROM 27, the control system 22 may be configured to control fluid flow as a function of the therapeutic material.

Some embodiments have a printable circuit board (PCB) equipped with a wireless interface (e.g., Bluetooth antenna) or a hardware connection configured to communicate the pump 18 and/or control system 22. The external catheter 14 can be configured to time out after a certain period, capture data, and communicate back and forth with the control system 22 or other off-catheter or on-catheter apparatus to share system specifications and parameters. The intelligent flow catheter 14 can be designed with proprietary connections such that design of knockoffs or cartridges 26 (discussed below) can be prevented to ensure safety and efficacy of the CSF circuit 10 and accompanying processes.

In addition to the management logic, the external catheter(s) 14 also may have a set of one or more flow sensors and/or a set of one or more pressure sensors. Both of those flow sensors are shown generically at reference number 29, and may be located upstream or downstream from their locations in FIG. 1B. For example, the left sensor(s) 29 generically shown in FIG. 1B can be a flow sensor, pressure, or both a flow sensor and pressure. The same can be said for the right sensor(s) 29 generically shown in FIG. 1B. They preferably are positioned between the ports 16 on the body and the remaining components as shown.

The flow sensor(s) 29 may be configured to detect flow through the bore of the catheter body, while the pressure sensor(s) 29 may be configured to detect pressure within the bore of the body. Among other functions, the flow sensor(s) 29 may monitor flow rate of fluid through the conduit bore and/or total flow volume through the conduit bore.

The catheter 14 preferably is configured to have different hardness values at different locations. Specifically, illustrative embodiments may use a mechanical pump 18, as shown and noted above. The pump 18 may periodically urge a compressive force along that portion of the catheter 14 it contacts at its interface 18A with the catheter 14. The outlet of the pump 18 in this case may be the portion of the catheter 14 that is receiving the output of a neighboring compressed catheter portion (e.g., a portion that is adjacent to the compressed catheter portion(s). To operate efficiently, illustrative embodiments form the catheter 14 to have a specially configured hardness at that location (e.g., 25-35 Shore A). Diameter also is important for flow and thus, one skilled in the art should determine appropriate diameters as a function of performance and durometer/hardness. Preferably, the catheter portion that contacts the pump 18 is softer than the remainder of the catheter 14, although both could have the same hardness. Accordingly, the catheter preferably has a variable hardness along its length and may even have a variable diameter.

Alternative embodiments may provide an open-loop CSF fluid circuit 10. For example, the CSF fluid circuit 10 may have an open bath (not shown) to which fluid is added and then removed.

Illustrative embodiments are distributed to healthcare facilities and/or hospitals as one or more kits. For example, one more inclusive kit may include the internal and external catheters 12 and 14. Another exemplary kit may include just the internal catheters 12 and the ports 16 (e.g., for a hospital), while a second kit may have the external catheters 14 and/or a single-use syringe and/or removable filter cartridges that can filter out toxic components, such as cancer cells, Tau, Abeta, TDP-43 from the CSF. Other exemplary kits may include the external catheters 14 and other components, such as the management system 19 and/or a CSF treatment cartridge 26 capable of removing toxic components. See below for various embodiments of the CSF circuit 10 and exterior components that also may be part of this kit.

Accordingly, when coupled, these pumps 18, valves (discussed below and all valves generally identified by reference number 28), internal and external catheters 14, and other components may be considered to form a fluid conduit/channel that directs CSF to the desired locations in the body. It should be noted that although specific locations and CSF containing compartments are discussed, those skilled in the art should recognize that other compartments can be managed (e.g., the lateral ventricles, the lumbar thecal sac, the third ventricle, the fourth ventricle, and/or the cisterna magna). Rather than accessing the ventricle and the lumbar thecal sac, both lateral ventricles could be accessed with the kit. With both internal catheters 12 implanted, CSF may be circulated between the two lateral ventricles, or a drug could be delivered to both ventricles simultaneously.

In illustrative embodiments, the CSF management system 19 generally manages fluid flow to target anatomy through the CSF circuit 10. To that end, that management system 19 has at least one pump 18 that directs flow of the CSF, and optionally at least one pump 18 that directs flow of a therapeutic material (e.g., a drug) though the CSF circuit 10 to desired anatomy. Alternative embodiments may have more pumps 18 for these functions or combine pumps 18 for these functions. The management system 19 also has a plurality of valves 28 to control flow, and the control system 22, as noted, is configured to control the pumps 18 to selectively apply the drug-carrying CSF to desired local anatomy. FIG. 1A also shows a user interface 20 that enables a clinician to control drug and fluid parameters in the CSF circuit 10 (discussed below) via the control system 22. In some embodiments, the management system 19 can automatically control the flow rate by reducing, stopping or accelerating the pump 18 as function of concentration of cancer cells and/or toxic proteins in the CSF after filtration.

Some embodiments may use a monitoring process, such as real-time spectroscopy, to monitor one or more of cancer cell concentration, toxic protein concentration and drug concentrations in the CSF. In some of these embodiments, a spectrophotometric sensor may be placed in the CSF circuit 10 to measure the localized concentration of a substance of interest, such as cancer cell or toxic protein or drug concentration based on its absorption at various wavelengths. For example, some embodiments may use a sensor constructed to measure a single wavelength or multiple wavelengths. The reading taken by the sensor may be relayed to the control system 22, where it would then be stored or processed for various purposes. This signal could be processed for a number of purposes, such as to trigger the control system 22 to alter the fluid flow, flow direction, and/or frequencies of certain periodic flows of bodily fluids (e.g., CSF) to provide a more localized and efficient therapeutic application to a patient in real-time. It will be appreciated that the signal could also be stored or displayed such that the changes to flow, direction or frequencies of period flows could be adjust manually.

FIG. 1C shows a high-level surgical flow process that may incorporate the CSF circuit 10 of FIG. 1A in accordance with illustrative embodiments of the invention. It should be noted that this process is substantially simplified from a longer process that normally would be used to complete the surgical flow. Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

The process begins at step 100 by setting up the internal catheters 12 inside the patient. To that end, step 100 accesses the ventricles and thecal sacs using standard catheters and techniques, thus providing access to the CSF. Step 102 then connects access catheters 12 to peritoneal catheters 12, which are tunneled subcutaneously to the lower abdomen. The tunneled catheters 12 then are connected at step 104 to the ports 16 implanted in the abdomen.

At this point, the process sets up an extracorporeal circulation set (i.e., the external catheters 14, or the “smart catheters” in some embodiments). To that end, step 106 may prime and connect the extracorporeal circulation set 14 to the subcutaneous access ports 16. Preferably, this step uses an extracorporeal circulation set, such as one provided by Enclear Therapies, Inc. of Newburyport, MA, and/or the external catheters 14 discussed above. The process continues to step 110, which connects an infusion line or other external catheter 14 to the management system 19, and then sets the target flow rate and time. At this point, setup is complete, and treatment may begin (step 112).

The process then removes endogenous CSF from the ventricle. This CSF may then be passed through a digestion region (e.g., through a cartridge 26 having a specific digesting material) where certain target proteins in the CSF are digested and/or a filtration region where cancer cells are removed from the CSF. For example, the cartridge 26 may have an inner plenum space 1830 of the cartridge 26 filled with a plurality of (e.g., porous, chromatography resin) beads that have been compression packed. To prevent constituents from entering or escaping from the cartridge 26, a filter membrane may be disposed at the first end of the cartridge 26 and a second filter membrane may be disposed at the second end of the cartridge 26.

At step 116, the treated CSF exits the digestion/filtration region and is returned via the CSF circuit 10 to the lumbar thecal sac. The process concludes at step 118, which stops the pump 18 when treatment is complete. The management system 19 then may be disconnected and the ports 16 flushed.

Any location providing access to the drug may be considered to be an input for the drug. In accordance with illustrative embodiments, the CSF circuit 10 is configured to improve the likelihood of the drug passing through the blood-brain barrier. To that end, the management system 19 enables the user or logic to independently set both the flow rate of CSF circulation (e.g., between 0.05 ml/min and 2.0 ml/min, such as 0.5 ml/min) and the dosing rate of the drug (e.g., between 0.01 ml/min to 2.0 ml/min, such as 0.02 ml/min). Preferably, these rates are different, although they can be the same. In illustrative embodiments, the CSF circulation rate is controlled to be different from the natural CSF flow rate. Note that the natural CSF flow rate is the rate of CSF flow without intervention by outside equipment, such as the pumps 18 and other CSF circuit components-even if it is within a range of typical non-interventionally controlled CSF flow rates. Thus, unless the context dictates otherwise, the non-natural CSF flow rate is the flow rate with such intervention. In other embodiments, the CSF flow rate is simply changed from its truly natural flow rate-i.e., the rate at which the CSF flows without intervention.

In some applications, the ameliorating agent may be decorated on the beads. In some applications, the cartridge 26 may be compression packed with a chromatography resin (e.g., agarose, epoxy methacrylate, amino resin, and the like) that has a protease covalently bonded (i.e. immobilized) to the three-dimensional resin matrix. The selected protease may be configured to degrade and/or removing target toxic proteins by way of proteolytic degradation. The resin may be a porous structure having a particle size commonly ranging between 75-300 micrometers and, depending on the specific grade, a pore size commonly ranging between 300-1800 Å. Preferred pore sizes ranges from 6-8 µm. Generally, cancer cells have sizes that range from 15-25 µm. Thus, at a high level, the cartridge 26 has one or more of ameliorating agent that removes and/or substantially mitigates the presence of cancer cells and/or toxic proteins from the CSF.

In one embodiment, a species non-specific filter, such as a size or ionic filter, is utilized. Even so, two species with similar physicochemical properties may be discriminated by their biological recovery rates if they are different.

In another embodiment, a multistage size filter is incorporated such that the target component is separated in the retentate, and then passed as a permeate in a second stage, but then discarded, whereas the retentate and the first permeate are joined and reintroduced to the body. In this way, a ‘notch’ filter may be constructed. This protocol may be combined with the rate of recovery protocol described above. Thus, two similarly sized components may be discriminated by their biological recovery rates. Furthermore, with this additional filtering step, the target component can be the longest recovery time species of the ‘pass-band’ cohort.

In yet another embodiment, a tangential filter is utilized. In another embodiment, continuous chromatographic processes, such as periodic countercurrent chromatography (PCC), multicolumn countercurrent solvent gradient purification (MCSGP), and continuous countercurrent tangential chromatography (CCTC), or single-pass tangential flow filtration (SPTFF) is utilized. In another embodiment, an enzymatic digester is utilized. The enzyme may be specific to the target component or may be somewhat non-specific.

It is instructive to consider that if a size cut-off filter which removes the target protein whilst also removing a critical protein, such as albumin is continuously employed with a high flow rate that deleteriously both the target and critical protein levels will be reduced. Similarly, if the repetition rate and duty cycle of the filtration process is too high again, both the target and critical protein levels will be reduced.

In one embodiment, CSF is caused to circulate within the subarachnoid space. In another embodiment, a repetition rate and duty cycle are established for a given treatment protocol. In yet another embodiment, the CSF is ameliorated by various means, such as filtering or digestion according to the established protocol. In one embodiment, the continuous CSF flow through the treatment device is adjusted to produce effective repetition rate and duty cycle levels.

In one embodiment, the rate of regeneration liquorpheresis protocol is as described below. Each component in the CSF has an equilibrium mechanism and rate of recovery. If a component is by some means removed from the CSF, the population will recover, or tend to recover, after some time. Therefore, if the component is again removed before the population is reestablished, the new long-term average equilibrium level for that component is reduced from its original. If, however, the component recovers with a quick rate, then the population reestablishes to the original level, or close to the original level, before the process by which the component was removed can once again act. If the component is a pathological entity, there may be no equilibrium. Thus, by choosing the repetition rate or duty cycle of the filtering mechanism with consideration of the various recovery rates of the CSF components a given component, or class of components, may be discriminated against and filtered out. Explicitly, in one embodiment, a size cut-off rejecting the target components is chosen. Further, a repetition time short compared to the target recovery time, but long compared to the non-target component recovery time is chosen.

For example, albumin is an abundant CSF protein (67 kDa) replenished by transport from the blood plasma pool, which in turn is supplied by the liver. Because it plays a central role in maintaining oncotic pressure, the homeostasis regulation is fast. Similarly, ions (Cl, Mg, Na ~18 Da) will have a fast rate of recovery. Neurotoxic peptides and proteins, such as amyloid-beta (4 kDa), tau (55-62 kDa), DPRs, and TDP-43 (45 kDa) migrate into the CSF over the long progression course of neurodegenerative disease. Therefore, filtering the CSF volume with a MWCO of <3000 Da once a day should remove the long-term population of the neuropeptides but allow a relatively unperturbed albumin (and salt) level. By adjusting the effective depth (MWCO) of filtering, the repetition rate, and duty cycle, the equilibrium CSF component populations can be manipulated. Similarly, a continuous filtering protocol may be adjusted by the filtration fluid flow rate.

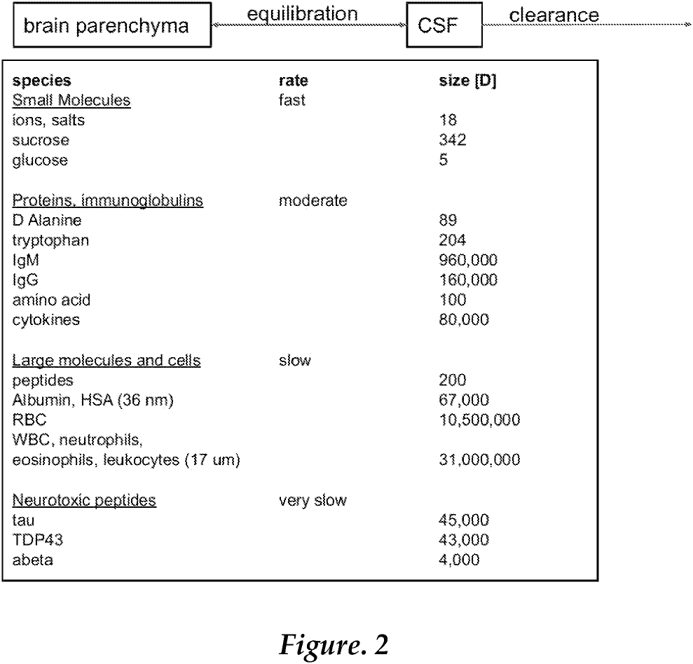

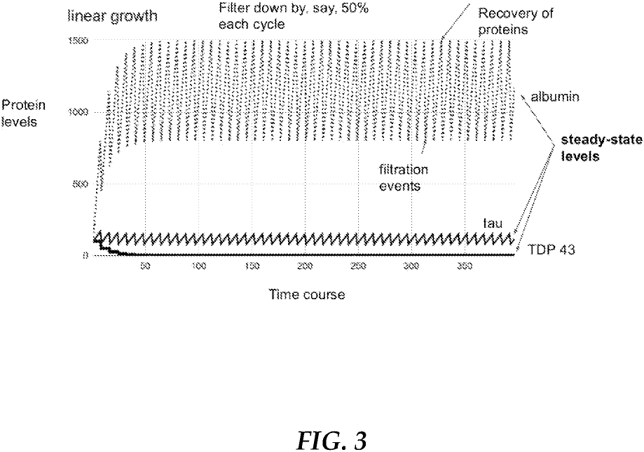

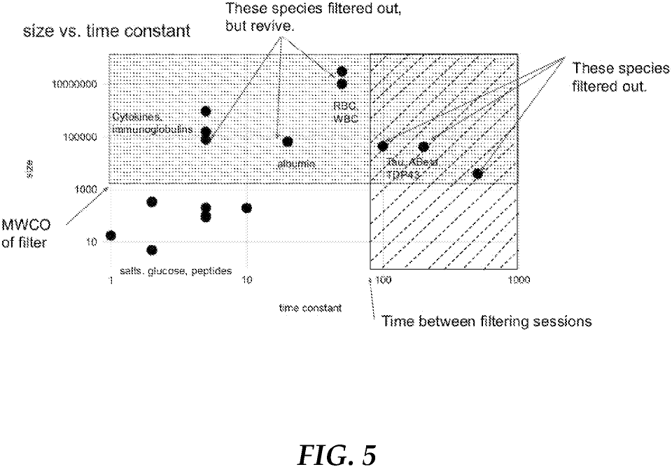

FIG. 3 illustrates a schematic predicting how three example CSF components, TDP-43, tau, and albumin undergo periodic filtration reduction (by say 50%), but achieve different steady-state levels depending on their regeneration rates. The examples from FIG. 2 are illustrated in graphical form in FIG. 5.

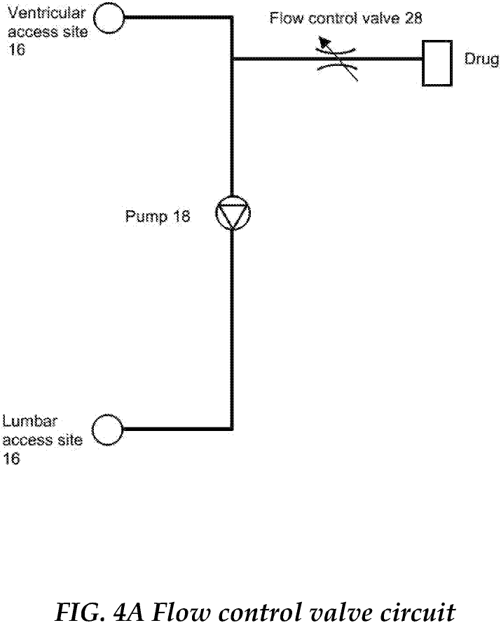

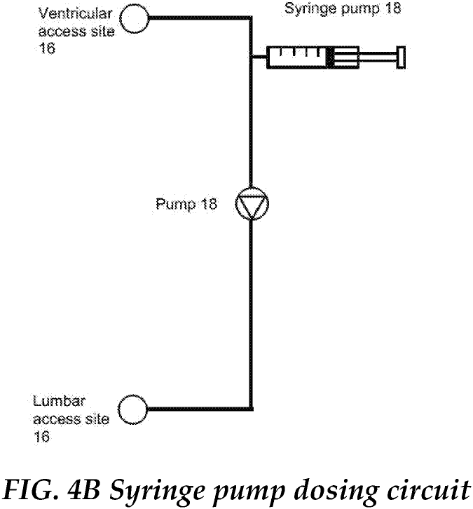

FIG. 4A schematically shows another embodiment in which a flow control valve 28 is used in place of Pump 2. In this embodiment, that flow control valve 28 preferably is programmed to control the dosing rate (i.e., the rate of adding the drug to the CSF circuit 10 carrying the CSF.

FIG. 4B schematically shows another embodiment in which a syringe pump is used inject a therapeutic drug into CSF by introducing it into the fluid line configured and usable with illustrative embodiments.

FIG. 5 is a schematic predicting how three classes of CSF components respond to the rate of regeneration filtration: 1) small molecular weight components (salts, glucose, peptides) pass through the filter (set here at 3000 MWCO); 2) larger molecular weight components (cells, albumin, immunoglobulins) with short regeneration times are reduced at each filtration step, but regenerate quickly, resulting in a high equilibrium concentration; and 3) large neurotoxic peptides and proteins (tau, Abeta, pathologic TDP-43) are initially size filtered, and do not recover since their regeneration rate is presumably smaller than the effective suppression rate due to the periodic size filtration, resulting in a low equilibrium concentration.

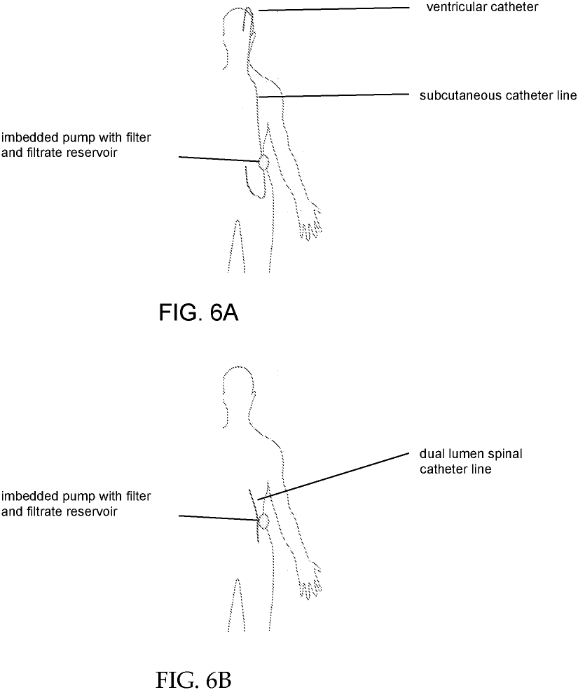

Exemplary fluid paths of CSF circulation past filtration element are shown in FIGS. 6A-B. In a multi-lumen embodiment, as shown, the conditioned endogenous CSF is then returned back to a CSF space in a different location than from which it was drawn. The second location or distal port for outflow or output is at a sufficiently different location from the first location or proximal port for inflow or input to create mixing of the conditioned and unconditioned CSF through a majority of the CSF space. For example, at least about 50%, 60%, 70%, 80%, 90% of the conditioned and unconditioned CSF in the CSF space can be mixed. The inflow and outflow ports usually can be at least two vertebrae apart, for example, if both ports are in the spinal area.

In some embodiments of the methods, the CSF circulation system may include one or more catheters, a reservoir, one or more valves, an anti-siphon device, connecting pieces, and a CSF filtering component. More details about each of these structures are enumerated below:

1) a proximal catheter (inserted proximally into the cerebral ventricles, cisterns, and interstitial or subarachnoid spaces and linked distally to a second cavity). Many possible types and configurations are available, the mostly widely used of which are the straight and right angle ventricular catheters. This catheter may be multi-perforated or fenestrated in order to optimize CSF or interstitial fluid (ISF) flow through it. In other embodiments, the proximal tip is comprised of multiple slits or multiple holes.

2) a reservoir, which may be attached to or part of the valve (optional). When present, the reservoir may be used to assess the patency of the shunt and to access the CSF for injections and/or samples if needed or desired. Contemplated approaches may have system components placed under the epidermis for sterility against infection. The systems may provide subcutaneous ports or reservoirs for CSF sampling, drug administration, or refreshing the ameliorating agent, or for removing neurotoxins and/or the accumulated circulating tumor cells (CTCs). These ports may resemble Ommaya reservoirs or subcutaneous venous access ports.

3) a unidirectional valve (or anti-reflux valve, i.e., one which prevents the flow of CSF towards the ventricles once the fluid has passed through the valve). Many types and configurations of these are available. The valve, whose function is to control the direction and rate of flow and is placed between the two catheters. The valves are designed to work at different pressures, depending on the patient, in order to provide optimal drainage of CSF and intracranial pressure. In one embodiment, the valve is programmable to allow for non-invasive adjustment of the opening pressure. In one embodiment the valve is comprised of a ruby ball and seat and stainless steel spring components in addition to the silicone exterior.

4) an anti-siphon device, which may be optionally attached to the valve. The anti-siphon device allows sudden increases in the differential pressure between the proximal and distal parts of the shunt to be corrected for instance when subjects move from lying down to standing up.

5) a distal catheter (linked proximally to the valve and inserted distally into the peritoneal cavity or into the entrance to another fluid filled cavity with the body, e.g., the intrathecal sac, or the right atrium of the heart in the case of a shunt configuration). Again, many types and configurations are available. The distal tip is usually open and may be multi- perforated in order to further facilitate the flow of CSF into the peritoneal (or cardiac) cavity.

6) connecting pieces, such as tubes, which connect the catheter segments outlined above. The tubes are generally made of silicone but can be made of other types of materials. The catheter segments comprising both the proximal and the distal components are typically comprised of silicone. They may be impregnated with antibiotics, such as rifampin and clindamycin/minocycline.

7) a CSF filtering component. In one embodiment, the device has one or more filters, either for cells and/or for other biologic materials or debris. In another embodiment, the valve and filters are positioned between the proximal and distal catheters.

Systems are constructed from materials which have been shown to be well tolerated by the body, such as silicone, polysulphone and stainless steel or other alloys such as titanium as well as a ruby ball to be used for the valve component.

Depending on a number of factors, the CSF flow rate may be greater than the rate of drug infusion, while in other embodiments, the CSF flow rate is less than rate of the drug infusion rate. Other embodiments may set them to be equal. In some embodiments, the CSF flow rate is higher than the natural flow rate during filtration of cancer cells. In some embodiments, the CSF flow rate is slowed down from the prior higher rate to ensure better filtration of cancer cells . Those skilled in the art can select the appropriate flow rate based on a variety of factors, including the drug being delivered, the illness, patient profile, rated pressure of the CSF circuit 10, etc.

The inventors recognized that varying the two rates in a coordinated manner enables more control of the filtration of cancer cells and optionally the concentration of drug being dosed. Stated another way, these two independent flow rates enable setting of the dosing rate, which allows the user to optimize filtration of cancer cells and/or drug concentration. At the same time, having the ability to set the flow rate allows the user to control the rate of delivery of drug and/or filtration of cancer cells (as opposed to relying upon natural CSF flow).

The selected CSF flow rate may be constant or variable. For example, the CSF flow rate may be set to a first rate for a first period of time, a second rate for a second period of time, and a third rate for a third period of time. As such, various embodiments enable flow of the CSF within the CSF circuit 10 at two or more flow rates at two or more different times. The drug delivery rate may be constant or variable in a similar manner but coordinated with the CSF flow rate to deliver preferred results. The rate at which cancer cells get filtered is dependent on the flow rate and the concentration of the cancer cells in the filtrate is continuously monitored using spectroscopic means to determine whether the flow rate needs to be increased or stopped depending on whether the concentration of cancer cells reaches or crosses a threshold value. Such spectroscopic means include but not limited to Infrared (IR) Spectroscopy, Ultraviolet-Visible (UV/Vis) Spectroscopy, Mass spectrometry, Nuclear Magnetic Resonance (NMR) Spectroscopy, Raman Spectroscopy and X-Ray Spectroscopy.

The extent of filtration, as reflected by the concentration of the target biomolecules, such as cancer cells in the retentate or filtrate, may be detected through a variety of means. These include optical techniques (e.g., Raman, coherent Stokes, and anti-Stokes Raman spectroscopy; surface enhanced Raman spectroscopy; diamond nitrogen vacancy magnetometry; fluorescence correlation spectroscopy; dynamic light scattering; and the like) and use of nanostructures, such as carbon nanotubes, enzyme linked immunosorbent assays, surface plasmon resonance, liquid chromatography, mass spectrometry, circular proximity ligation assays, and the like.

For example, the photonic energy of terahertz wave is in the same order of magnitude as the rotational and vibrational energy levels of organic and biological macromolecules, so it has unique advantages in detecting cells and biological macromolecules. Wei shi et al. has developed a set of terahertz single measurement system based on the tilt wave front of grating pulse technique. The system was employed for the terahertz detection of trace living cervical cancer cells. The characteristic absorption peaks were identified by Lambert-Beer law, respectively, at 0.49, 0.71, 1.04, 1.07, 1.26 and 1.37 THz. The absorbance is proportional to the cell concentration. Thus one can use transient terahertz spectroscopy to detect the presence of live cancer cells in biological samples, such as CSF. (Wei Shi, Detection of living cervical cancer cells by transient terahertz spectroscopy, Journal of Biophotonics, Volume14, Issue1, January 2021.)

Baird et al. has disclosed a method for the detection of circulating tumor cells (CTC) using mass spectrometry (MS), through reporter-ion amplification. Particles functionalized with short-chain peptides are bound to cells through antibody-antigen interactions. Selective release and MS detection of peptides is shown to detect as few as 690 cells isolated from a 10 mL blood sample. Enumeration of CTCs is done by immuno-recognition with antibodies conjugated to particle clusters carrying peptide mass labels that are selectively released after CTC isolation and quantified by MALDI-TOF-MS. The signal ion emission reactive release amplification (SIERRA) method can be used to cluster nanoparticles carrying peptide MS labels to magnetic particles conjugated to antibodies, thereby amplifying the number of MS labels carried per antibody in order to reach lower detection limits in the count of CTCs. (Zane Baird, Tumor Cell Detection by Mass Spectrometry Using Signal Ion Emission Reactive Release Amplification, Anal. Chem. 2016, 88, 14, 6971-6975 Publication Date:Jun. 28, 2016).

Personalized cancer treatment relies on the detection of actionable genomic aberrations in tumor cells. CSF management system described herein provides for a unique way of sampling, characterizing the circulating cancer cells and obtaining real time information on how the cancer cells react when exposed to a therapeutic drug and what type of mutations in the cancer cells are more susceptible to the treatment with a drug or a combination of drugs. In some embodiments, the CSF management system can identify the type of receptor markers in the circulating tumor cells in the CSF as to whether they are wild type or mutant using detection means, such as flow cytometry, and provide information as to whether a particular subset of tumor cell is more or less receptive to the drug being administered, such as Methotrexate. Thus, the CSF management system can provide personalized cancer treatment for patients by allowing sampling of CTCs in the CSF, identifying and characterizing the CTCs, determining which subtype of cancer cells are more receptive to a particular drug treatment.

Likewise, Raman spectroscopy has been used to observe uptake, metabolism and response of single-cells to drugs. Photodynamic therapy is based on the use of light, a photosensitizer and oxygen to destroy tumor tissue. Pablo et al. used single-cell Raman spectroscopy to study the uptake and intracellular degradation of a novel photosensitizer with a diphenylacetylene structure, DC473, in live single-cells from colorectal adenocarcinoma cell lines. (Julia Gala de Pablo, Detection and time-tracking activation of a photosensitiser on live single colorectal cancer cells using Raman spectroscopyAnalyst, 2020, 145, 5878-5888). One can use any of the aforesaid spectroscopic methods to detect, quantitate the concentration of cancer cells in CSF and determine the variable value.

In some embodiments, the amount or concentration of circulating cancer cells in the CSF is determined by analyzing the retentate from the filter. In some embodiments, the filter module is removable filter cartridge which can be analyzed to determine the concentration of cancer cells that have been removed from the CSF due to filtration. The concentration of cancer cells in the retentate and/or the removable filter cartridge can be determined by using detection means as described above which includes spectroscopic methods such as flow cytometry in combination with microfluidics and/or mechanical sedimentation methods, such as centrifugal separation. The concentration of cancer cells in the retentate and/or the removable filter cartridge enables one to determine when and if the next round of filtration should occur.

Amelioration may include the use of a treatment system (e.g., UV radiation, IR radiation), as well as a substance, whose properties make it suitable for amelioration. Amelioration of CSF or ameliorated CSF - which terms may be used interchangeably herein - refers to a treated volume of CSF in which one or more target compounds have been partially, mostly, or entirely removed. It will be appreciated that the term removed, as used herein, can refer not only to spatially separating, as in taking away, but also effectively removing by sequestering, immobilizing, or transforming the molecule (e.g., by shape change, denaturing, digestion, isomerization, or post-translational modification) to make it less toxic, non-toxic or irrelevant.

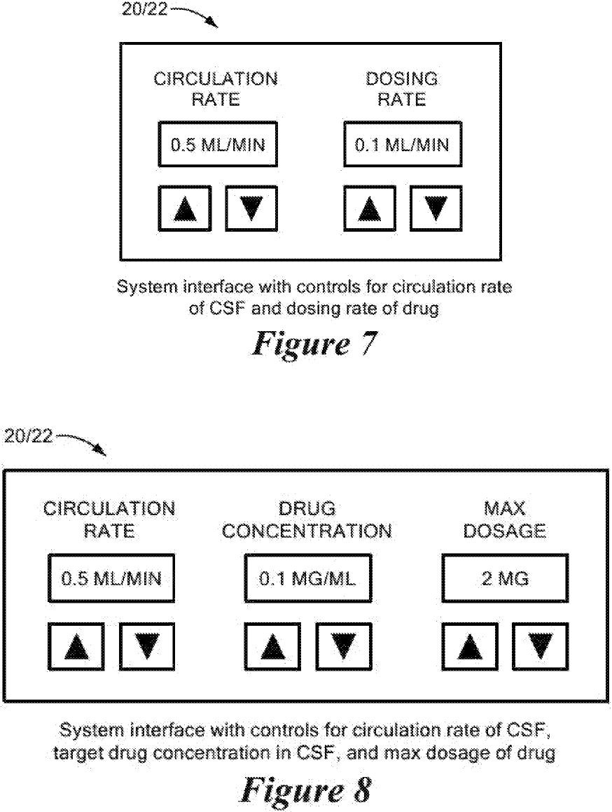

FIGS. 7 and 8 schematically show two different user interfaces 20 in accordance with illustrative embodiments. Rather than requiring the user to input a dosing rate, however, the user may specify a drug concentration and the management system 19 responsively may adjust the dosing rate accordingly to achieve that concentration. The user can also input a maximum dosage. After this dosage was reached, the management system 19 would automatically stop treatment. FIG. 8 shows one such interface 20 (e.g., a graphical user interface or a manual interface).

It should be noted that the during actual processing, the CSF flow rate may differ at different parts of the CSF circuit 10- total CSF flow rate in the CSF circuit 10 is not necessarily homogenous. For example, some parts of the CSF circuit 10 may be wider (e.g., certain human geographies) and thus, may be slower than the average CSF circuit flow rate, while other portions may be narrower, causing a nozzle effect and increasing the CSF flow rate at that point. Near the pump 18 (e.g., at the pump outlet), however, the CSF flow rate can be controlled to provide a desired rate across the entire CSF circuit 10, even if that rate may deviate in local parts of the CSF circuit.

The discussion above relates to delivering a therapeutic material, such as a drug, over a longer infusion period (e.g., 5 minutes, 10 minutes, 30 minutes, 1-6 hours, days, etc.).

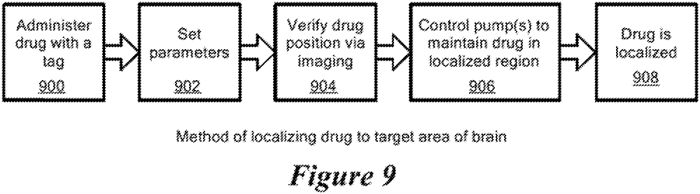

FIG. 9 shows another embodiment that localizes drug delivery at a target area of the brain using a bolus drug infusion. Specifically, as known by those in the art, a dose of drug can be delivered in a short time period (e.g., 10 seconds, 20 seconds, 60 seconds), or over a longer period (i.e., gradual drug administration, as noted above). The shorter drug delivery is known in the art as a “bolus” drug delivery.

Specifically, to optimize delivery, FIG. 9 alternates the flow direction of the pump 18. The pump 18 thus has programmable controls, via the control system 22, for flow rate and frequency of these alternations. The flow rate and frequency preferably are programmed to achieve a desired delivery profile.

In a manner similar to the other process discussed above, the process of FIG. 9 is substantially simplified from a longer process that normally would be used to complete the localize drug delivery. Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

FIG. 9 therefore delivers a drug intrathecally using positive displacement at a desired flow rate. It may incorporate the components discussed above, as well as principles discussed for other embodiments, such as that discussed above with regard to FIGS. 1 & 4. The process of FIG. 9 begins at step 900 by adding the drug in a bolus dose to the CSF circuit 10, and/or administering a tag for imaging to the drug. In the latter example, its position can then be tracked using standard imaging techniques to determine when the drug has reached the target anatomy. Alternative embodiments add the drug to the CSF without administering a tag. Such steps may use other techniques to ensure the drug is localized at the desired target anatomy.

Step 902 sets the desired flow rate, direction, timing, and other parameters for the CSF circuit 10 to accomplish the bolus application. For example, specific computer program code on a tangible medium within the control system 22 may cooperate with other components of the CSF circuit 10 to control addition of the therapeutic material, localize the therapeutic material, or both. Next, after step 904 verifies the position of the drug at a target anatomy, step 906 controls the pump 18 to maintain the drug at that target location. Among other ways, step 906 may control the pump(s) 18 to oscillate at a desired flow rate and frequency to contain the drug at that prescribed or desired anatomical location for a pre-set period of time.

After the bolus reaches the target anatomy, the pump 18, which can be programmable and/or have logic, can reverse CSF flow; specifically, the pump 18 can alternate quickly between pushing and pulling flow of the CSF so that the bolus of drug is localized to the target anatomy in the brain (or another target anatomy). In other words, the higher concentration of drug in a portion of the CSF can moved back and forth over the target region. Other embodiments can simply slow down the CSF flow rate to ensure a longer drug application to the target. Either way, these embodiments preferably “soak” the target with the drug, providing a higher quality drug administration. As a result, despite using less of the drug than would be administered by prior art systems, this embodiment still administers a desired amount of the drug to the target by this localizing technique, consequently minimizing toxicity and drug costs (step 908).

It should be noted that “reaching” the target anatomy may be defined by the user or other entity within the control system 22. For example, the portion of CSF in the CSF circuit 10 having the higher concentration of drug (from the bolus) may be considered to have reached the target anatomy when some identifiable portion of it (e.g., the highest concentration, or an interior point within the spread of the drug in the CSF) may be within a prescribed distance upstream of the target, or a prescribed distance downstream of the target. Some embodiments may require the defined portion of CSF with the high drug concentration to actually be at or in contact with that target region. Other embodiments may consider the drug to have “reached” the target simply by calculating the time it should take to reach that area, using artificial intelligence/machine learning, and/or through empirical studies.

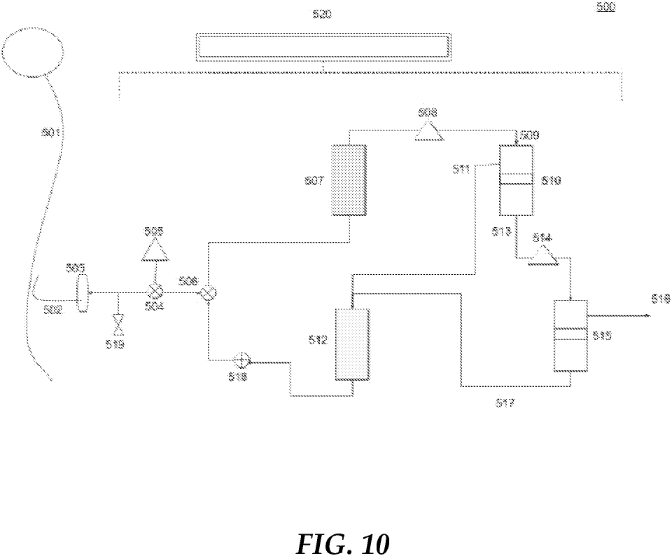

FIG. 10 illustrates one embodiment of a system 500 that withdraws in a batch processing fashion cerebrospinal fluid from the subarachnoid space 501 of a subject by means of a lumbar catheter 502. The lumbar catheter 502 is connected to a subcutaneous port 503. When a treatment session begins, cerebrospinal fluid is extracted through a needle to a fluid circuit beginning with a three-way valve 504. Untreated fluid is withdrawn through a three-way valve 504 by a syringe pump 505 and ejected through the three-way valve 506. To prime the untreated fluid reservoir 507 cerebrospinal fluid is initially withdrawn at a slow rate (e.g., 2-100 mL/hr). This rate is sufficiently slow as to allow the subarachnoid space pressure to remain near equilibrium values. Once a first volume (e.g., 20-200 mL) is obtained, the withdrawing from the subject is paused. The fluid in the reservoir 507 is then pumped by a pump 508 to the inlet 509 of a tangential flow filter 510. The pump 508 is a peristaltic pump. In some embodiments it is a syringe pump.

The filter 510 has a filter pore that measure 7.5 to 8 µm in diameter sufficient to retain cancer cells but pass the target protein species. The retentate 511 is directed to a reservoir 512 for treated fluid. The reservoirs 507 and 512 are in one embodiment flexible bladders, and contain valves for purging, and safety release. The permeate 513 containing the target protein species is directed by another pump 514 to another tangential flow filter 515. The pump 514 is a peristaltic pump. In some embodiments it is a syringe pump. The molecular weight cutoff (e.g., 3 kDa) of this filter 515 is sufficient to retain the target protein species but pass smaller analytes, such as glucose and ions. The retentate 516 containing the target protein species of this second filter 515 is directed to waste. The permeate 517 is directed to the reservoir 512 for treated fluid.

After a volume is accumulated in the reservoir 512 for treated fluid, the fluid is directed through valve 506 and valve 504 by pump 505 back to the subcutaneous port 503. The fluid then is reintroduced into the subject’s subarachnoid space 501 by way of the subcutaneous port 503 and lumbar catheter 502. In one embodiment, the acts are substantially sequential. However, after the initial priming, treatment, and filling of the treated fluid reservoir 512, the next filling of the untreated reservoir 507 occurs soon before or after the discharging of the treated fluid reservoir 512. In this manner, the aspiration and injection of volumes of cerebrospinal fluid will not cause the subject to bear a large volume change in the subarachnoid space for any significant amount of time.

In one embodiment, the intake stroke of syringe pump 505 withdraws fluid from the subject, while the output stroke feeds the untreated fluid reservoir 507. A following intake stroke withdraws fluid from the treated fluid reservoir 512, while the output stroke returns fluid to the subject. A time delay between the acts of aspirating from and injecting to the subject is implemented to allow the newly treated fluid to be transported to the cranial vault. In some embodiments this delay is 20 min. This delay allows the newly aspirated fluid to be substantially different from the recently injected treated fluid. The coordination of these acts is controlled by a control system 520 which is connected to the various valves, pumps, and sensors. The control system comprises a user interface, a communications subsystem, programmable memory, and alarms. The pressure in the fluid circuit is monitored by a pressure sensor 519 close to the aspiration and injection port 503. An access port 518 is provided to sample fluid to determine the concentration behavior of the component of interest, to make other diagnostic determinations, and to deliver drugs. In one embodiment, treatment comprises multiple sets of multiple cycles of injection and aspiration with equilibrium time between sets to allow for recovery of non-targeted cerebrospinal fluid components.

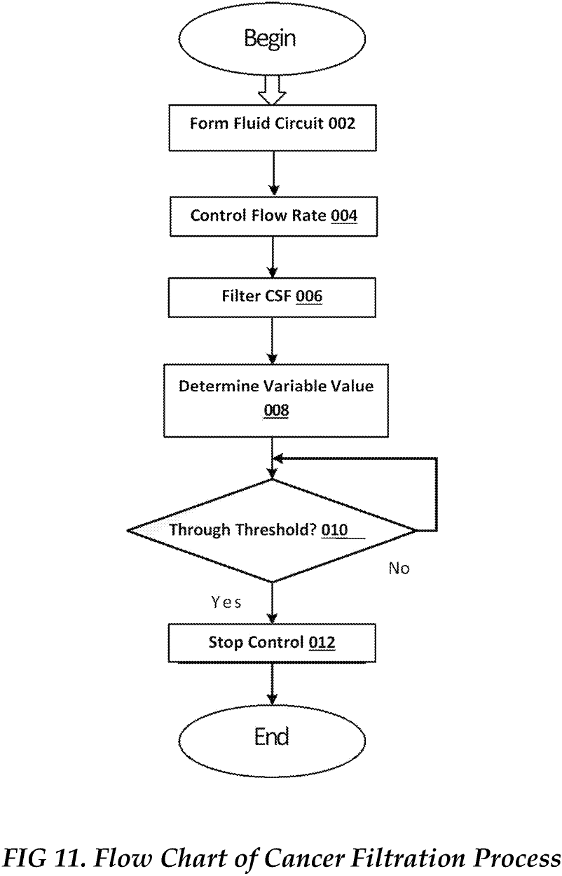

FIG. 11 shows a flow chart of cancer filtration process. It should be noted that this process is substantially simplified from a longer process of FIG. 10. Accordingly, the process of filtering the cancer cells has many steps, such as testing steps, forming, purging, controlling the flow, filtering, sampling, determining variable value, comparing with threshold value and accelerating, decelerating or shutting the flow which those skilled in the art likely would use. In addition, some of the steps may be performed in a different order than that shown, or at the same time. Those skilled in the art therefore can modify the process as appropriate. Moreover, as noted above and below, many of the structures noted are but one of a wide variety of different materials and structures that may be used. Those skilled in the art can select the appropriate materials and structures depending upon the application and other constraints. Accordingly, discussion of specific materials and structures is not intended to limit all embodiments.

The process of FIG. 11 begins at step 002 where the fluid circuit is first formed using the components of the CSF management systems, such as pumps, catheters, ports and controllers. The next step 004 controls the flow rate of the system to ensure that the flow is different from that of the natural flow rate. In some embodiments, the flow rate of the system is higher than the natural CSF flow rate. In some embodiments, the flow rate of the system is lower than the natural CSF flow rate. In some embodiments, the flow rate of the system is the same as the natural CSF flow rate. The step 006 indicates the filtration of the CSF to remove cancer cells, the filtered CSF is then sampled to determine the variable value as noted in step 008. Once the variable is determined, it is compared with the threshold value as in step 010 to determine whether to continue the process of filtration or to stop the process of filtration as noted by step 012. If the variable value is less than the threshold value, then the process of filtration is continued with the flow rate in the system being higher than the natural CSF flow rate. If the variable value is greater than the threshold value, then the controller sends a signal to stop the pump which shuts off to ensure that the flow rate of the system returns to the natural CSF flow thereby slowing or stopping the filtration process.

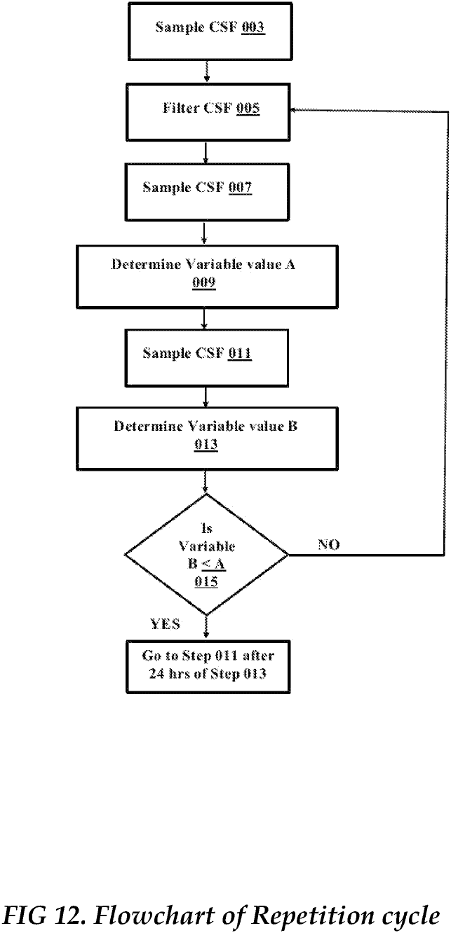

FIG. 12 shows a flow chart of how one would determine the repetition cycle or frequency of treatment using the CSF management system. The process begins in Step 003 as shown in the FIG. 12. A sample of the CSF is collected from the patient before the start of the filtration process. The amount of cancer cells in the sample before treatment is determined using spectroscopic means as described elsewhere. The CSF is then filtered in step 005 following the methods described above. A sample of CSF is collected post filtration and amount of cancer cells in the sample is determined using spectroscopic means. In step 009, the variable value A is determined as a function of concentration of cancer cells in the samples before and after filtration. A CSF sample is retrieved from the patient 24 hours post treatment in Step 011. The amount of cancer cells in the sample is determined in using spectroscopic means. In step 013, the variable value B is determined as a function of concentration of cancer cells in the samples from step 007 and step 011. In step 015, the value of variable B is compared with that of variable A.

If the amount of cancer cells in CSF sample at step 011 is greater than the amount of cancer cells in CSF sample at step 007, then it implies that the cancer cells have regenerated or infiltrated into the CSF from other parts of the body post filtration. Therefore, the process is repeated from step 005 where the CSF is again filtered to remove the regenerated cancer cells.

If the amount of cancer cells in CSF sample at step 011 is lower or equal to the amount of cancer cells in CSF sample at step 007, then it implies that the cancer cells have not yet regenerated or infiltrated into the CSF at the time of measurement. Therefore, the process is stopped, and the patient is sent home. A fresh CSF sample is collected at step 011 after 24 hours. Then step 013 is repeated to determine how much time, such as how many days it takes for the amount of cancer cells in the sample to become greater than the amount of cancer cells in the CSF immediately post filtration. The number of days thus determined provides the frequency or repetition cycle of treatment for the patient.

For instance, if the cancer cells are found in the CSF after four days post filtration but not after three days post filtration, then the CSF filtration process can be repeated after three days or the frequency of treatment/CSF filtration is every three days. Likewise, if the cancer cells are found in the CSF after 7 days post filtration and not after six days post filtration, then the CSF filtration process can be performed once a week. Thus, the method and accompanying CSF management system provides a dynamic way for actively monitoring the efficacy of filtration process of removing the cancer cells from the CSF and provides a way to set the repetition cycle of how often one needs undergo the CSF filtration to reduce the level of cancer cells and thereby lower the risk of brain metastasis thereby providing personalized treatment schedule for each individual patient.

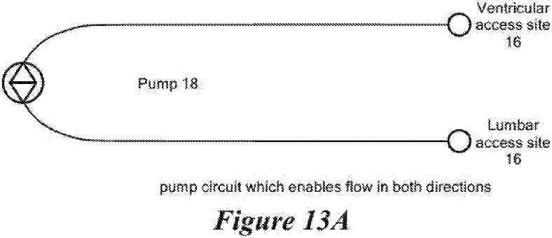

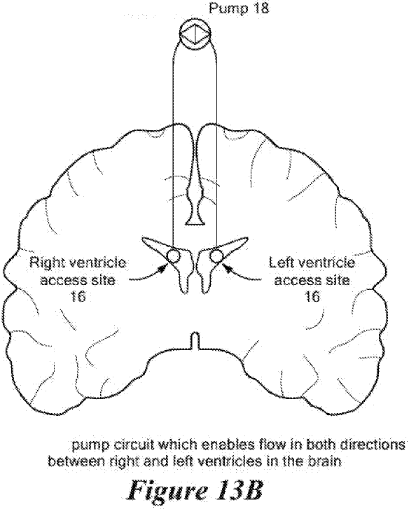

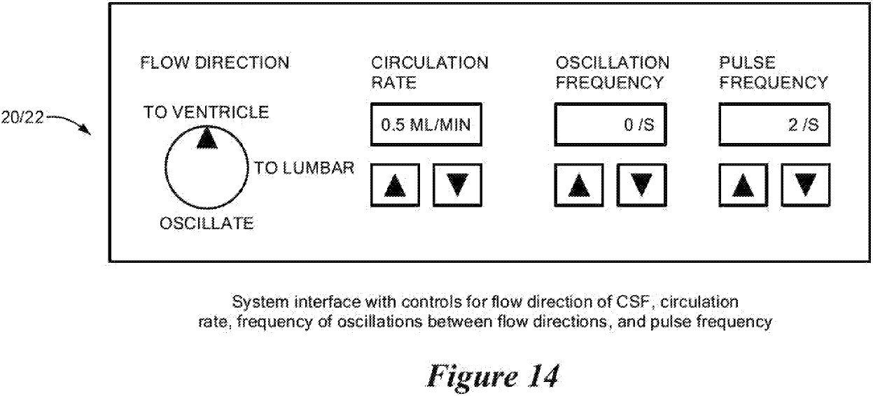

Illustrative embodiments can be implemented in a number of different manners with catheters 12/14, pumps 18, valves 28, etc. similar to those discussed above (including the noted external catheters 14). FIGS. 13-14 show several exemplary implementations.

Flow direction oscillation and a pulsatile flow pattern could also be produced using a bidirectional pump 18 instead of using pinch valves (e.g., FIG. 13A and FIG. 13B). The pump 18 can be programmed to switch flow directions at a frequency set by the user. While flowing in one direction, the pump 18 can be programmed to pulse by starting and stopping at a frequency also set by the user. Those skilled in the art may use other techniques to provide bidirectional flow.

In addition to those noted above, some embodiments may set the frequency, flow rate, and other parameters as a function of the requirements and structure of the anatomy and devices used in the treatment (e.g., in the CSF circuit 10). Among others, those requirements may include the diameter of the catheters in the CSF circuit 10, physical properties of the drug, the interaction of the drug at the localized region, the properties of the localized region, and other requirements and parameters relevant to the treatment. Those skilled in the art may select appropriate parameters as a function of the requisite properties.

FIG. 14 schematically shows another system interface 20 configured in accordance with illustrative embodiments. Specifically, whether controlling delivery parameters by pinch valve, a bidirectional pump 18, or other means, the delivery profile can be controlled manually with an interface 20, such as the interface 20 shown in FIG. 14, and/or a delivery profile loaded onto the management system 19. As with the other interfaces, this interface 20 may be a fixed control panel, or a graphical user interface on a display device.

Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

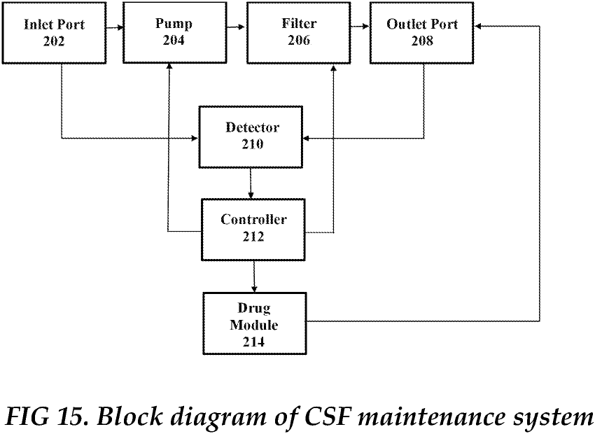

FIG. 15 shows a block diagram of one embodiment of the CSF maintenance system. It should be noted that FIG. 15 only schematically shows its components. Those skilled in the art should understand that each of these components can be implemented in a variety of conventional manners, such as by using hardware, software, or a combination of hardware and software, across one or more other functional components. Accordingly, the representation of the FIG. 15 and other components in a single box is for simplicity purposes only. In fact, in some embodiments, such as pumps, filter, detector and drug delivery modules of the figure can be distributed across a plurality of different parts or devices and not necessarily within the same housing or module. It should be reiterated that the representation of FIG. 15 is a significantly simplified representation of an actual CSF maintenance system. Those skilled in the art should understand that such a device may have many other physical and functional components. Accordingly, this discussion is in no way intended to suggest that FIG. 15 represents all of the elements of a CSF management system.

In one embodiment shown in FIG. 15, there are two sample ports one at inlet port (202) and one at outlet port (208). The inlet port (202) and the outlet port (208) are in fluid communication with the pump (204) and an optional filter (206) by means of catheter which is not shown in the block diagram for simplicity. The inlet port (202) allows the user to sample the CSF from the patient before filtration. The outlet port (208) allows the user to sample the CSF post filtration. The inlet port (202) and the outlet port can be in communication with an optional detector (210) which uses spectroscopic means to determine the concentration of target components of interest, such as cancer cells and/or toxic proteins and marker components, such as red blood cells or albumin in the CSF before and after filtration. The detector (210) is in communication with the controller (212) which determines whether to accelerate the flow or decelerate the flow or stop the flow of the CSF by communicating with the pump (204). The controller makes the decision based on whether the concentration of cancer cells and/or toxic proteins in the CSF post filtration has reached or passed a threshold value. Optionally the controller (212) can also decide based on other variables, such as concentration of marker components, such as albumin or red blood cells in the retentate or permeate post filtration. The controller (212) can also be in communication with the drug delivery module (214), which can optionally inject a drug, such as methotrexate into the filtered CSF, which is then returned to the patient. The controller (212) can also be coupled with the optional filter (206), which can filter, reduce, mitigate or neutralize toxic components, such as cancer cells, bacteria, virus and/or toxic proteins, such as Tau,Abeta, TDp-43 using one or more of biochemical, physiochemical, affinity, enzymatic, mechanical means as described in the disclosure. The controller (212) can optionally control the flow rate and filtration by communicating with the pump (204) and filter (206).

Additional details of amelioration are taught by PCT Application No. PCT/US20/27683, filed on Apr. 10, 2020 and PCT/US19/042880, filed Jul. 22, 2019, the disclosure of each is incorporated herein, in its entirety, by reference. In a similar manner, details for devices are taught by USSN 17/489,620 filed on. Sep. 29, 2021 (published as US 2022-0096743) the disclosure of which is incorporated herein, in its entirety, by reference.

Definitions: As used in this description and the accompanying claims, the following terms shall have the meanings indicated, unless the context otherwise requires.

As used herein, the term “patient” refers to any mammal, man, animal, or ex vivo tissue. The mammal can be a non-human mammal, a non-human primate or a human. In some embodiments, the mammal is a domestic animal (e.g., canine, feline, rodentia, etc.), an agricultural mammal (e.g., bovine, ovine, equine, porcine) or a laboratory mammal (rodentia, rattus, murine, lagomorpha, hamster) or a non-domesticated mammal. The patient may be suffering from a particular disease or may be at risk of suffering from a disease.

As used herein, the term “CSF space” refers to any volume of cerebrospinal fluid found in the cranial or spinal areas that is in contact with any component of the nervous system and may or may not be within the tissue. Interstitial fluid may also be targeted for removal or toxic substances as described above.

As used herein, the phrases “conditioning CSF” or “conditioned CSF” interchangeably refer to CSF wherein one or more target compounds and/or cancer cells have been partially, mostly or entirely removed. It will be appreciated that “removed,” as used herein, can refer not only to spatially separating, as in taking away, but also effectively removing by sequestering, immobilizing, or transforming the molecule (either by shape change, digestion, isomerization, or post-translational modification) to make it non-toxic or irrelevant. It will be appreciated that the phrases “ameliorating CSF” or “ameliorated CSF” can be used interchangeably with the phrases “conditioning CSF” or “conditioned CSF” throughout the disclosure.

As used herein, the term “CNS” means central nervous system.

As used herein, the term “CSF” means cerebrospinal fluid.

As used herein, the term “conditioning” means modifying the composition or state of the fluid, either by digestion, removal, or filtration or inactivation of substances, or by changing the physical state of the fluid. It will be appreciated that the terms “conditioning” and “ameliorate,” as defined above, can be used interchangeably throughout the disclosure.

As used herein, the term ‘neurological disease’ means any condition affecting the CNS or CSF.

As used herein, the term “regeneration rate” means a rate by which a composition, or quantity, of a component within a substance is restored.

As used herein, the term “toxic protein” refers to protein aggregates, protein fibrils, peptide aggregates, exosomes that are believed to cause pathology and neurological diseases. For instance, Tau protein aggregates and Amyloid beta protein aggregates are found in patients with Alzheimer’s disease. Likewise TDP-43 protein aggregates are found in patients with FTD and dementia. Examples of toxic proteins include but not limited to dipeptide repeat proteins, beta amyloid protein and Tau protein.

As used herein, the term “AD” means Alzheimer’s disease.

As used herein, the term “Abeta” means amyloid beta peptide.

As used herein, the term “CTE” means chronic traumatic encephalopathy.

As used herein, the term “ALS” means amyotrophic lateral sclerosis.

As used herein, the term “T3DM” means type 3 diabetes.

As used herein, the term “TBI” means traumatic brain injury.

As used herein, the term “PSP” means progressive supranuclear palsy.

As used herein, the term “PD” means Parkinson’s disease.

As used herein, the term “CTE” means chronic traumatic encephalopathy.

As used herein, the term “PSP” means progressive supranuclear palsy.

As used herein, the term “HD” means Huntington’s disease.

As used herein, the term “MS” means multiple sclerosis.

As used herein, the term “IMD” means intracranial metastatic disease.

As used herein, the term “FTD” means frontotemporal dementia.

As used herein the term “cartridge” can refer to a more general object operable to ameliorate the patient’s fluids.

As used herein the term “filter” or “filtering” refers to filtering, removing, retarding, neutralizing, mitigating cancer cells and/or toxic biomolecules, such as Tau, Abeta, TDp-43 etc. from CSF to provide therapeutic relief to a patient.

### Filter

In some embodiments, the CSF is ameliorated by a filter or cartridge containing a transforming agent. This treatment may be episodic or continuous. The entire system then has the effect of creating a high clearance rate of cancer cells from the brain parenchyma. In another aspect of the invention, the CSF is ameliorated by a filter or cartridge containing a transforming agent to encourage cancer cells and/or toxic proteins from the interstitial space to migrate directly into the CSF. Once in the CSF, the circulating system will remove, degrade cancer cells. The entire system then has the effect of a high clearance of cancer cells from the brain parenchyma. In some embodiments, the filter is of the type: dead end filter, tangential filter, continuous chromatographic processes, such as periodic countercurrent chromatography (PCC), multicolumn countercurrent solvent gradient purification (MCSGP), and continuous countercurrent tangential chromatography (CCTC), or single-pass tangential flow filtration (SPTFF), or combination thereof.

In one embodiment, the filtration method used by the filters is based on one or more of:
1) electromechanical methods including, but not limited to: radiofrequency, electromagnetic, ultraviolet radiation, acoustic wave, piezoelectric, electrostatic, nano-, molecular/biologic force, atomic force, and ultrasonic filtration and ultrafiltration based methods;
2) biochemical/physicochemical properties and/or temperature methods including, but not limited to: size-exclusion, pore flow, solution diffusion, protein size or secondary, tertiary or quaternary structure, diffusion, hydrophobic/hydrophilic, anionic/cationic, high/low binding affinity, chelator, magnetic or nanoparticle-based systems, and various neurochemical filtration systems; and
3) bi-specific affinity methods including, but not limited to, specific antibodies, immunotherapy-based, immuno-modulatory, ex-vivo immunotherapy using immobilized antibodies or antibody fragments, nucleic acids, receptors, anti-bacterial, anti-viral, anti-DNA/RNA, protein/amino acid, carbohydrate, enzymes, isomerases, compounds with high-low bi-specific binding affinity based systems.

In some embodiments, the filtration is based on enzymatic digestion through the use of one or more enzymes that may include: trypsin; elastase; clostripain; calpains, including calpain-2; caspases, including caspase-1, caspase-3, caspase-6, caspase-7, and caspase-8; M24 homologue; human airway trypsin-like peptidase; proteinase K; thermolysin; Asp-N endopeptidase; chymotrypsin; LysC; LysN; glutamyl endopeptidase; staphylococcal peptidase; arg-C proteinase; proline-endopeptidase; thrombin; cathepsin, including the cathepsins E, S, B, K, or L1; Tissue Type A; heparinase; granzymes, including granzyme A; meprin alpha; pepsin; endothiapepsin; kallikrein-6; kallikrein-5; and combinations thereof.

In some embodiments, the filtration is based on affinity methods relying on cell surface markers of the group: EpCAM, HER2+, EGFR, heparinase, and Notch1.

Exemplified disease conditions treatable by illustrative CSF processing systems and methods include, but are not limited to: Cerebral Vasospasm with or without subarachnoid hemorrhage, Guillain Barre Syndrome (GBS), Alzheimer’s disease (AD), mild cognitive impairment, prodromal Alzheimer’s disease, Parkinson’s disease (PD), Huntington’s disease (HD), Multiple Sclerosis (MS), Amyotrophic Lateral sclerosis (ALS), Spinal Cord Injury, Progressive supranuclear palsy (PSP), Frontotemporal dementia (FTD), Traumatic Brain Injury (TBI), Chronic Traumatic Encephalopathy (CTE), Type 3 Diabetes Mellitus (T3DM), Lewy body dementia, Lupus, Stroke, cancer affecting the brain or spinal cord, including but not limited to leptomeningeal carcinoma, intracranial metastatic disease (IMD), or primary brain tumors (such as gliomas, including glioblastomas, astrocytomas, oligodendrogliomas, and ependymomas), meningiomas, pituitary adenomas, vestibular schwannomas, and primitive neuroectodermal tumors (medulloblastomas), prion disease, encephalitis from various causes, meningitis from various causes, diseases secondary to enzymatic or metabolic imbalances, pathological complications due to exposure to biological warfare, etc.

### Filtration Component

In some embodiments, the filtering component may include one or more of bi-specific affinity, biologic or immunoaffinity, cationic exchange, anionic exchange, hydrophobicity and size exclusion. For example, the filtering component can be a column or a cartridge placed with the hollow lumen of the catheter. In some embodiments, the catheter comprises the filtering component (e.g., bound to the inner surface of the catheter by covalent or non-covalent bonding).

With respect to size exclusion and filtration, the filtration component can be any type, e.g., membranous, nanoparticular, flat, tubular or capillary. In another embodiment, the system is coated with a biologic material that would capture any toxic agents. In another embodiment, a device that captures toxic agents (“capturing device”) is separate from but placed with the silicone shunt conduit. This capturing device may be a scaffold, cartridge or other device that allows for CSF sampling through it with the capture of undesirable toxic products. In another embodiment, the capture device is disposable and may be replaced as needed. In another embodiment, the capture device is part of the said shunt system and may be revised or replaced over time.

In one embodiment, a tangential flow filter is utilized. In another embodiment, continuous chromatographic processes, such as periodic countercurrent chromatography (PCC), multicolumn countercurrent solvent gradient purification (MCSGP), and continuous countercurrent tangential chromatography (CCTC), or single-pass tangential flow filtration (SPTFF) is utilized.

With the recent advances in nanotechnology and ultrafiltration, it is now possible to remove agents on the nanometer scale as well as those on the micrometer scale, offering nearly a 1000x improvement in targeted filtration than previous systems. Prior filtration methods based on size were limited to 0.2 micron filters allowing the majority of smaller toxic molecules to pass directly through the filter and back to the patient.

In some applications, in addition to introducing an enzymatic ameliorating agent into the CSF (e.g., in the SAS), the ameliorating agent also may be circulated through the CSF, rather than remaining stationary or substantially stationary. In some implementations, the ameliorating agent may be completely implanted (e.g., via a catheter) in, for example, the SAS and circulated within the SAS; while, in other implementations, the ameliorating agent may be implanted within the body of the mammalian subject but outside of the SAS, from whence the ameliorating agent may be circulated into and back out of the SAS. In another embodiment, when an ameliorating agent is introduced without circulation, the CSF may exit the SAS, be circulated past the ameliorating agent, and returned to the SAS. In yet another embodiment, both the CSF and the ameliorating agent is circulated.

The antibodies or other therapeutic, could alternatively be affixed to the inner lumen of the catheter tubing either along the entire length of the system or for some pre-specified length. In either instance, there would be the possibility of replacing or re-charging the components that capture the toxic substances. The use of biologic separation (including Abeta and Tau/pTau proteins in AD, alpha-synuclein in PD, etc.) can be beneficially applied to a number of diseases by altering the neuro-immune axis using a platform ex- vivo immunotherapy approach.

In some embodiments, amelioration of biomolecules within the CSF may also be by enzymatic digestion or transformation, such that the ameliorating agent modifies or degrades the biomolecule in the CSF. To that end, an enzyme-substrate pair may be selected by means of a panel and counter panel search. For example, the panel of candidate enzymes to digest the biomolecules may be graded for stability, commercial availability, and relevant mechanism of interaction, while the counter panel may ensure that candidate enzymes would not affect substances in the CSF that the enzyme should not alter. Alternatively, the enzyme may be discovered through a microbial screen, such as a mutant hunt or a nitrogen vitality assay. In yet another embodiment, the enzyme may be selected through a biomolecule engineering computational model. Alternatively, a method for proteolytic degradation using PROTAC (PROteolysis TArgeting Chimera) which involves a heterobifunctional molecule with a covalent linker that connects two active domains. This is a method that is capable of removing specific unwanted proteins by inducing selective intracellular proteolysis. (See Qi, S. et al./ (2021). PROTAC: An Effective Targeted Protein Degradation Strategy for Cancer Therapy. Frontiers in Pharmacology, 12.)

Some methods of ameliorating and the amelioration chemistry are described in U.S. Provisional Application Number 62/702,186, filed on Jul. 23, 2018; U.S. Provisional Application Number 62/702,188, filed on Jul. 23, 2018; U.S. Provisional Application Number 62/702,191, filed on Jul. 23, 2018; International Application Number PCT/US2019/042879; and International Application Number PCT/US2019/042880; as well as U.S. Provisional Application Number 62/960,861, filed on Jan. 14, 2020, the disclosures of which are hereby incorporated herein by reference in their entireties.

In some embodiments, the agent for use in the ameliorating system may include: trypsin; elastase; clostripain; calpains, including calpain-2; caspases, including caspase-1, caspase-3, caspase-6, caspase-7, and caspase-8; M24 homologue; human airway trypsin-like peptidase; proteinase K; thermolysin; Asp-N endopeptidase; chymotrypsin; LysC; LysN; glutamyl endopeptidase; staphylococcal peptidase; arg-C proteinase; proline-endopeptidase; thrombin; cathepsin, including the cathepsins E, S, B, K, or L1; Tissue Type A; heparinase; granzymes, including granzyme A; meprin alpha; pepsin; endothiapepsin; kallikrein-6; kallikrein-5; and combinations thereof.

Other features can be added to the filter system including a differential centrifugal force to aid in the rapid separation of items of interest, e.g. ultrafiltrate, proteins, cells, etc.

In some embodiments, filtering removes about 1% of cancer cells and/or number of related or distinct toxic biomolecules (e.g., protein, peptide, oligopeptide) from the CSF. In some embodiments, filtering removes about 5% of cancer cells and/ or number of related or distinct toxic biomolecules (e.g., protein, peptide, oligopeptide) from the CSF. In some embodiments, filtering removes about 10% of cancer cells and/or number of related or distinct toxic biomolecules (e.g., protein, peptide, oligopeptide) from the CSF. In some embodiments, filtering removes about 20% of cancer cells and/or number of related or distinct toxic biomolecules (e.g., protein, peptide, oligopeptide) from the CSF. In some embodiments, filtering removes about 25% of cancer cells and/or number of related or distinct toxic biomolecules (e.g., protein, peptide, oligopeptide) from the CSF. In some embodiments, filtering removes about 30% of cancer cells and/or number of related or distinct toxic biomolecules (e.g., protein, peptide, oligopeptide) from the CSF. In some embodiments, filtering removes about 40% of cancer cells and/or number of related or distinct toxic biomolecules (e.g., protein, peptide, oligopeptide) from the CSF. In some embodiments, filtering removes about 50% of cancer cells and/ or number of related or distinct toxic biomolecules (e.g., protein, peptide, oligopeptide) from the CSF. In some embodiments, filtering removes about 75% of cancer cells and/or number of related or distinct toxic biomolecules (e.g., protein, peptide, oligopeptide) from the CSF. In some embodiments, filtering removes about 80% of cancer cells and/or number of related or distinct toxic biomolecules (e.g., protein, peptide, oligopeptide) from the CSF. In some embodiments, filtering removes about 90% of cancer cells and/or number of related or distinct toxic biomolecules (e.g., protein, peptide, oligopeptide) from the CSF.

Various embodiments may remove ranges between any of the above noted percentages. For example, some embodiments may remove between 10% and 90%, 30% and 80%, 20% and 90%, etc. of cancer cells and/or number of related or distinct toxic biomolecules (e.g., protein, peptide, oligopeptide) from the CSF. For efficiency, each range is not expressly spelled out here, but should be considered included.

In some embodiments, the filtering comprises removing pathological cells (e.g., B-cell, T-cells, macrophages, erythrocytes and other blood cells, cancer cells) and cellular debris. In one embodiment, a system of catheters accessing the CSF provides circulation and fluid contact of the CSF with an agent operable to identify CTCs, for instance an agent based on cell surface markers, such as EpCAM, Notch1, HER2+, EGFR, heparanase, or Notch1, and to selectively sequester them, to reduce the occurrence of intracranial metastatic disease. In one embodiment, the active agent is immobilized on a solid substrate, which substrate is placed in fluid contact with the CSF or serum. In one embodiment, the CSF fluid is passed through a spatial filter operable to select CTCs based on their size, shape, or flexibility. In one embodiment, the CSF fluid is passed through a spatial filter operable to select CTCs based on their physicoelectric properties.

In some embodiments, a cartridge-based schema can be employed for rapid changing or combinations of the aforementioned purification-based schema. For example, a system combining size, antibody and charge based approaches is envisioned with single or multiple cartridges for the purification, such that when the time came for replacement of the purification filter, antibody, etc., it could be done in an easy to use, rapid exchange system. The filtering system or chromatographic cartridges (e.g., bi-specific interaction, ionic exchangers, size exclusion) can be external or internal to a patient’s body. In some embodiments, the filtering cartridges or filters are contained within one or more lumens of the single or multi-lumen catheters. In some embodiments, the lumen of the catheters, or sections thereof, are coated (e.g., by covalent or non-covalent binding) with chromatographic moieties (e.g., bi-specific capture moieties, including antibodies and nucleic acids, cationic or anionic exchangers, hydrophobic moieties, and the like).

In some embodiments, the CSF is contacted with multiple substrates, e.g., combining size, bi-specific and charge-based selection criteria. The conditioning step can be performed external or internal to a patient’s body. In some embodiments, the conditioning substrates are contained within one or more lumens of the multi-lumen catheters. In some embodiments, the lumen of the catheters, or sections thereof, is coated (e.g., by covalent or non- covalent binding) with chromatographic moieties (e.g., bi-specific capture moieties, including antibodies and nucleic acids, cationic or anionic exchangers, hydrophobic moieties, and the like).

### Qualifying Patients

Potential subjects for the procedure discussed below are scored based on regulatory clinical guidelines. The subjects may be identified as having the target CSF component and the corresponding disease by means of clinical measures that may or may not rely on direct CSF quantification and analysis. For example, the clinical Norris score or ALSFRS-R may be used to assess a person’s state with respect to ALS; ADAS-cog for Alzheimer’s disease, etc. For example, one can use one or more of cytogenetic analysis, Immunophenotyping, Liquid biopsy, Sputum cytology, Tumor marker tests, CT scan, MRI, Radionuclide scan, Bone scan, PET scan and Ultrasound to assess a person’s state with respect to cancer. Notwithstanding, CSF samples may be withdrawn and analyzed by liquid chromatography mass-spectrometry, BCA, ELISA, proximity ligation assays, flow cytometry, SiMOA or other antibody-based assays, before, during, and after treatment to surveil the efficacy of the procedure.

### Prophylactic Measures

In another embodiment, the procedure is employed on a patient population that is presymptomatic and is early in the disease progression with the thought that liquorpheresis may be preventative or substantially retarding especially in a high risk factor group, such as one having a genetic predisposition to the disease.

### Treatment Regimen

The rate of regeneration protocol, determined by the physiological behavior of the target components, may allow an episodic treatment schedule. Alternatively, a fully implantable, continuous device may be used if the amelioration rate is deemed sufficient.

In one embodiment, the system is deployed as a preventative measure against metastatic spread to the brain once a cancer is discovered elsewhere in the body by removing circulating tumor cells (CTCs) from the CSF. In another embodiment, the system is deployed chronically. In another embodiment, the system is deployed intermittently. For example, the system could be used just before or just after a resection of a tumor. The system is deployed with a filtration regimen operable to allow sufficient equilibrium concentrations of critical CSF components.

In one embodiment, the stopping criteria for the treatment is determined beforehand based on clinical need, patient tolerance, or clinical experience. For example, treatment will stop when the concentration of cancer cell is reduced by 90%, 80%, 70%, 60%, 50%, 40%, 30%, 20%, 10% or other percentage. Alternatively, treatment will stop when 1, 5, 10, 20, 50, 100, 1000 cycles or other number of cycles are performed. When the treatment begins, it may be extended or prematurely halted depending on clinical measures.

### Fluid Configurations

Some illustrative systems allow for a number of different CSF outflow connections for the processing of CSF between any point in the CSF system such that total outflow is relatively equal during at least most of the time it is operational. In illustrative embodiments, the system creates a dynamic circulation with significant mixing within the cranial or spinal CSF space. Some embodiments process large volumes of CSF in a short amount of time with minimal or no impact on the endogenous intracranial/intraspinal pressure and volume. The spatial location of the inflow and outflow ports are sufficiently distant to allow for CSF flow throughout a major portion or the entire CSF space. The custom cranial or spinal catheters can be introduced via a number of routes, including but not limited to: single ventricular insertion, dual ventricular insertion, cisterna magna, sub-arachnoid, single level spinal insertion, dual/multi- level spinal insertion and ventriculo-spinal.

The catheters may be single lumen, or multi-lumen. An advantage of the catheter system with two or more lumens as well as multiple holes for inflow and outflow along the length of the catheter not only minimizes clogging but provides greatly increased turnover and access to the basal cistern, ventricular, cranial as well as spinal subarachnoid CSF due to the greater efficiency at removing compounds of interest arising from less reprocessing of the same fluid. In some embodiments, a first catheter is inserted into a brain ventricle or into the cervical spine, and a second catheter is inserted into the lumbar spine. In addition, any of the above systems can be fashioned to exchange CSF from any two points within the subarachnoid space. One example is a ventricular catheter with entry/exit sites communicating with the subarachnoid space overlying the adjacent brain parenchyma. The epidural, subdural and interstitial spaces may also be accessed.

One exemplification of a multi-lumen embodiment includes use of a single level spinal insertion that is inserted in the lumbar space and fed cranially such that the catheter tip is in the cervical region. In this example, CSF inflow may be from the cervical portion and output from the lumbar portion and/or anywhere along the length of the multi-lumen catheter, depending on the number and location of exit ports along the outflow lumen. In another embodiment, inflow can be at the lumbar region and outflow at the cervical, subarachnoid or ventricular regions. In another embodiment, both the inflow and outflow ports are in the ventricular space, for example, with one port in a first ventricle and a second port in a second ventricle. In another embodiment, the inflow and outflow ports are located at different sides of the same ventricle.

In other embodiments, one of the inflow or outflow ports can be in the spine (e.g., sacral, lumbar, thoracic or cervical) and the other inflow or outflow port can be in the subarachnoid or ventricular space. In some embodiments, both the inflow and outflow ports are in the ventricular space, for example, where the inflow port is in a first ventricle and the outflow port is in a second ventricle (dual-ventricular embodiment). Depending on the design of the system, the quantitative distance between the inflow and outflow ports can be at least about 4 cm, for example, at least about 5 cm, 8 cm, 10 cm, 12 cm, 15 cm, 20 cm, 30 cm, 40 cm, 50 cm, or 60 cm, or longer, depending on the length of spine of an individual patient.

### Drug Delivery

In addition to removal of specific toxins from the CSF, some illustrative methods contemplate the delivery of therapeutic agents on the return cycle. That is, after a given volume is passed through the specific purification schema of interest, such as filtration that results in the removal of cancer cells from CSF, a specific pharmacologic agent or drug can be administered directly to the CNS and bypass the blood-brain barrier. This provides the opportunity for specific delivery of pharmaceuticals, such as Methotrexate to the CNS without the often many systemic side effects associated with oral or intravenous delivery. One of the challenges of drug delivery via the CSF is designing the drug to penetrate the brain/spinal cord parenchyma. A variety of ways including adjusting the hydrophilicity or using liposome-based approaches in conjunction with the system described herein may be envisioned. Thus, for the first time, the system described herein allows for the combined removal of cancer cells as well as delivery of specific therapeutic agents to the CNS.

Some illustrative methods also contemplate the infusion of artificial CSF fluid into the system, if needed, at any time. The combined purification of CSF with return of artificial CSF with appropriate physical/chemical safeguards in addition to the purified CSF is but one possibility. The system may also be primed with such a physiologically compatible artificial CSF solution.

Some illustrative systems allow for the active movement of a wide range of CSF volumes over time, and do not require the removal of CSF from the human body. Due to the varying entry and exit sites in the custom catheter, the system allows for the production of active, in addition to the normally passive, CSF flow. The active movement of CSF can be generated in a number of ways including but not limited to motorized pumps for active CSF withdrawal.

Furthermore, the pump system can have a variety of mechanisms that facilitate the requirement that inflow and outflow are relatively equal. Examples of suitable pumps include, but are not limited to, rotatory, syringe- driven, volumetric, peristaltic, piston, pneumatic, bellows, electromagnetic, magnetostrictive, hydraulic, nano- and biologic/molecular. The pumps can be a single apparatus with bi-directional functionality or two unidirectional pumps that are in communication with one another. There are several pumping mechanisms available to reach the desired endpoint of creating active, in addition to the normally passive, flow of CSF. Examples of suitable pumps include rotatory, syringe-driven, volumetric, peristaltic, piston, pneumatic, bellows, electromagnetic, magnetostrictive, hydraulic, and the like.

The pump can be external or internal to the patient’s body. Internal or implantable pumps are known in the art (e.g., an Archimedes screw pump). The rate of CSF flow could also be pre-determined through the use of a one-way valve that opens above a certain desired ICP and would not open if the ICP were below that value. These valves would also include an anti-siphon device so that there was not a difference in flow based on body position (e.g., supine versus standing).

In some embodiments, the system is implantable. For example, the shunt system is generally positioned under the skin with one end of the catheter placed within the ventricular system, ISF or subarachnoid space and is known as the ventricular or proximal end of the device. The other end is placed within the peritoneal cavity or alternatively within another fluid-filled space, such as a second subarachnoid space, the spinal canal, or in the right atrium of the heart, and is called the distal end of the device. In one embodiment, the shunt may have tubes of additional length that can be coiled within the body to create a greater surface area for contact of the contaminated CSF with the device.

With respect to some embodiments of the systems, the catheter assembly includes a single tubular member having an intake and an outlet lumen and port that allows for CSF/ISF to pass through and interact with the device. CSF/ISF can be sampled from the following CNS compartments: ventricle, cisterna magna, subarachnoid space, and interstitial space as well as the central canal and other spinal fluid spaces that surround the spinal cord and lumbar theca. The central fluid can be diverted to: all the above, as well as any cavity capable of accepting fluid, such as, but not limited to the peritoneal cavity, cardiac chambers, blood vessel, and urinary bladder. The fluid may also be recirculated back to the CNS compartments.

In some embodiments, the first location or proximal port is in the cranial subarachnoid space. In some embodiments, the first location or proximal port is in one or more ventricles or the interstitial space of the cisterna magna. In some embodiments, the second location or distal port is in the sacral, lumbar, thoracic or cervical subarachnoid, interstitial or CSF space. In some embodiments, the second location or distal port is in the lumbar CSF space, for example at S1, L5, L4, L3, L2, L1 or above or below. In some embodiments, the second location or distal port is in the peritoneal cavity.

In some embodiments, the first location or proximal port is in one or more ventricles. In some embodiments, the second location or distal port is in the peritoneal cavity. In some embodiments, the second location or distal port is in the right atrium of the heart.

In some embodiments, the first location or proximal port and the second location or distal port is in the ventricular space. For example, both the first location or proximal port and the second location or distal port can be on opposite sides of the ventricle, in another example, the first location or proximal port is in one ventricle and the second location or distal port is in another ventricle.

In some embodiments, the distance between the first location or proximal port and the second location or distal port is adjustable. For example, a pair of tubular members in a multi-lumen catheter can be axially adjusted relative to one another. In another embodiment, the system uses nano- or molecular or biologic motors to facilitate drainage of CSF/ISF.

In some embodiments, the CSF is removed or withdrawn at a flow rate in the range from about 0.04 ml/min to about 0.33 ml/min, for example, from about 2.5 ml/hour to about 20 ml/hour, In some embodiments, the flow rate is maintained by a pump that has a flow rate adjustable between about 0.04 ml/min to about 0.33 ml/min, for example, from about 2.5 ml/hr to about 20 ml/hr. In some embodiments, fluid flow is regulated by a valve, which can either be set to allow flow within at a particular range of pressures or routinely interrogated and flexibly adjusted to the flow and pressure desired. In another embodiment, the pump comprises a peristaltic pump that is isolated from the CSF flow. In some embodiments, the pump is implantable with an Archimedes screw.

In some embodiments, the volume of CSF removed is below the volume that would induce a spinal headache or symptoms of over drainage. In some embodiments, the volume of CSF removed from the patient never exceeds the rate of production.

In some embodiments, the distance between the first location and the second location is at least about 4 cm, for example, about 5, 10, 20, 25, 30, 35, 40, 45, 50, 55, 60, 70, 80, 90 or 100 cm. In some embodiments, the distance between the first location and the second location is separated by at least about 2 vertebrae.

The system allows for passive flow but may also include a mechanism of active pumping with transient or continuous flow. Furthermore, a number of measures (including, but not limited to, pressure sensor, velocity detector, bubble detector, pH, temperature, osmotic equilibrium, blood pressure, transmembrane pressure sensor) to ensure both control of CSF/ISF removal as well as patient safety are included.

Pressure sensors to continuously record/maintain/adjust intracranial and/or intraspinal pressures are also available. Programmable control of intake, output and overflow exhaust valves are additional contemplated features. The system is adjustable to a broad range of biologic parameters and flows. Alarms and automatic on/off settings are further included to provide a signal for immediate attention and interrogation of the system.

The system can be located at any portion of the system, although this is most typically envisioned near the proximal end of the system over the calvarium. A given volume of CSF removed from the patient’s brain at any one time is typically less than that which produces symptoms associated with spinal headache or over drainage.

The shunt can include two catheter tubes that are interconnected by a pressure control valve. In one embodiment, the valve is both one-way (unidirectional) and has an anti-siphon device. The proximal catheter, referred to as the ventricular catheter, has one end inserted through a hole that is drilled in the skull through which the catheter is placed into a ventricle (or cisterna magna) of the brain. The other end of the ventricular catheter is connected to the pressure control valve, which is typically implanted under the scalp. The second catheter, known as the drainage catheter, has one end connected to the pressure control valve, and the other end of the drainage catheter empties into a lower body cavity, usually the peritoneal cavity.

### Pressure Control

The pressure control valve is designed to open at a predetermined pressure to allow drainage of CSF from the ventricle of the brain to the peritoneal cavity, where it is re-absorbed by the body. This maintains the CSF pressure in the brain within a set range of values. The pressure control valve can be of a number of different designs. Some pressure control valves use flexible elastomeric membranes to flex open under pressure, while others use ball and spring designs or other means to open in order to control CSF pressure. In one embodiment, the valve can be interrogated by an external device that will allow the user to change the pressure range over which the valve is functional and hence control the rate of CSF flow. To reduce risk of infection, the valves are designed to allow CSF flow only out of the brain, and not back into the brain.

An additional effect occurs when CSF flows through the shunt, not because of a positive pressure in the brain, but due to the negative (suction) pressure created because the lower end of the drainage catheter is typically at a lower level then the end of the ventricular catheter in the brain, or because the egress pressure is less than the intracranial pressure. This effect is commonly known as the siphoning effect and causes CSF to drain even when the CSF pressure in the brain is within normal or set parameters. The siphoning effect varies with the position of the patient, being the most extreme siphoning effect occurs when the patient is upright, while there is virtually no siphon effect when the patient is lying down. In all situations, however, the siphoning effect is extremely undesirable and must be eliminated or kept to an absolute minimum. To control unwanted siphoning, the filtering systems are equipped with a second valve (a siphon control valve) placed in line with the pressure control valve in order to prevent or reduce siphoning. In another embodiment, the anti-siphon device could be part of the pressure control valve. The basic principle is generally one or more flexible walls of a chamber surrounding a port through which the fluid must flow to drain. The flexible walls are designed to collapse onto and occlude the port when negative pressure is experienced. This provides additional flow resistance to counteract the effect of the negative pressure, while still allowing drainage under positive pressure from the brain.

The flow rates may be varied and are limited by the pressure differential placed on the catheter walls, but generally can be in the range of 0.04 ml/min to approximately 0.33 ml/min, for example, about 2.5 to 20 ml/hr.

### Diagnostics

Illustrative embodiments contemplate the possible periodic re-use or re-charging of the filtration/processing component of the system. For instance, in the ex- vivo immunotherapy approach, a specific eluent can be used to release the captured oligomers or proteins and regenerate the active antigen binding sites on the antibodies. Furthermore, this eluted compound represents a purified human protein that can then be used as a “neuropharmaceutical” agent. For example, in Alzheimer’s disease, purified Aβ or Tau/pTau components may then be released and used for a variety of other commercial or research studies involving the structure-activity function and relationship of disease-specific compounds in human disease. Also, the ability to automatically or periodically collect CSF or specific subcomponents without resorting to a lumbar puncture and store/freeze creating a CSF bank for specific disease processes is contemplated.

In some embodiments, a CSF fluid conduit system, such as those discussed above, direct CSF flow to or from a patient having one or more ports 16 in fluid communication with the patient’s subarachnoid space. This system has a CSF fluid conduit that is compatible and specially configured for use with a CSF circuit having a pump 18 that drives CSF fluid flow.

To those ends, the system (or kit) includes a body with a fluid traversing bore having first and second ends. Preferably, the body is removably couplable between the port 16 of the patient and the pump 18 so that the bore is in fluid communication with both the port 16 and pump 18 when removably coupled there between. The body also is configured to form a closed loop CSF channel when removably coupled between the pump and the interface. The CSF channel and bore are in fluid communication with the patient’s subarachnoid space when the body is removably coupled.

In illustrative embodiments, the system also has a flow sensor 29 (or communicates with a flow sensor) configured to detect flow through the bore of the body, and a pressure sensor 29 configured to detect pressure within the bore of the body. The system also may communicate with and/or include a controller 27 having a communication channel with the pump 18. The controller 27 may be configured to determine a variable indicative of cancer cell concentration in the CSF and determine whether the variable has attained or passed through a threshold value. The controller 27 also may be configured to stop control of the CSF flow rate in response to the variable attaining the threshold value. In that case, the CSF flow rate may return to a natural CSF flow rate of the patient after stopping control of the CSF flow rate. Some embodiments of the system also may include a removable filter (e.g., a filter cartridge), and/or communicate with a filter (e.g., the filters noted above). The controller and system may implement one or more of the various other features discussed above.

It is understood that the examples and embodiments described herein are for illustrative purposes only and that various modifications or changes in light thereof will be suggested to persons skilled in the art and are to be included within the spirit and purview of this application and scope of the appended innovations.

Illustrative embodiments are distributed to healthcare facilities and/or hospitals as one or more kits. For example, one more inclusive kit may include the internal and external catheters 12 and 14. Another exemplary kit may include just the internal catheters 12 and the ports 16 (e.g., for a hospital), while a second kit may have the external catheters 14 and/or a single-use syringe. Other exemplary kits may include the external catheters 14 and other components, such as the management system 19 and/or a CSF treatment cartridge 26. Various embodiments of the CSF circuit 10 and exterior components described above may also be part of this kit.

Accordingly, when coupled, these pumps 18, valves, internal and external catheters 14, and other components may be considered to form a fluid conduit/channel that directs CSF to the desired locations in the body. It should be noted that although specific locations and CSF containing compartments are discussed, those skilled in the art should recognize that other compartments can be managed (e.g., the lateral ventricles, the lumbar thecal sac, the third ventricle, the fourth ventricle, and/or the cisterna magna). Rather than accessing the ventricle and the lumbar thecal sac, both lateral ventricles could be accessed with the kit. With both internal catheters 12 implanted, CSF may be circulated between the two lateral ventricles, or a drug could be delivered to both ventricles simultaneously.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A method of managing cancer in a patient’s cerebrospinal fluid (“CSF”), the method comprising:
forming a fluid circuit comprising fluidly communicating a set of catheters with the patient’s brain ventricle and the patient’s lumbar;
controlling the flow rate of the CSF through the CSF fluid circuit;
filtering cancer cells from the CSF through the fluid circuit;
determining a variable indicative of cancer cell concentration in the CSF;
determining that the variable has attained or passed through a threshold value; and
stopping control of the CSF flow rate in response to the variable attaining the threshold value, the CSF flow rate returning to a natural CSF flow rate of the patient after stopping control of the CSF flow rate.

2. The method as defined by claim 1 wherein controlling the flow rate comprises increasing the CSF flow rate to be greater than the natural CSF flow rate.

3. The method as defined by claim 1 wherein the fluid circuit comprises a pump used to control the flow rate of the CSF through the CSF fluid circuit, further wherein stopping comprises turning off the pump.

4. The method as defined by claim 1 wherein the fluid circuit comprises a filter through which the CSF passes.

5. The method as defined by claim 4 wherein the filter comprises one or more of an electromechanical filter, a mechanical filter, a biochemical property filter, a physiochemical property filter, a temperature filter, and/or a bispecific affinity filter.

6. The method as defined by claim 1 wherein the variable comprises the concentration of the cancer cells in the CSF.

7. The method as defined by claim 1 wherein determining a variable comprises calculating the variable from information about the CSF.

8. The method as defined by claim 7 wherein the variable includes the concentration of a prescribed type of cell in the CSF.

9. The method as defined by claim 1 wherein determining a variable comprises detecting or determining an approximate or near exact concentration of cancer cells in the CSF.

10. The method as defined by claim 1 wherein determining a variable comprises using mass spectroscopy to determine the concentration of a prescribed cell type within the CSF.

11. The method as defined by claim 1 further comprising adding a therapeutic material to the CSF after filtering.

12. The method as defined by claim 1 further comprising:
obtaining a first sample of the CSF before filtering;
obtaining a second sample of the CSF within 60 minutes after stopping control of the CSF flow rate; and
obtaining a third sample of the CSF at least one day after obtaining the second sample;
formulating a filtering plan as a function of the difference between the first, second and third samples.

13. The method as defined by claim 1 further comprising performing forming, controlling, filtering, determining a variable, comparing the variable with a threshold value, and stopping in sequence as a set of acts on each of a plurality of days, determining the number of days between each set of acts as a function of the concentration of the variable at the end of each set of acts.

14. The method as defined by claim 1 further comprising dynamically varying the flow rate of the CSF through the CSF fluid circuit as a function of the determined indication of cancer cell concentration in the CSF.

15. The method as defined by claim 1 wherein the fluid circuit comprises a filter cartridge through which the CSF flows.

16. The method as defined by claim 1 wherein determining the variable comprises determining a relationship between albumin and cancer cells in the CSF.

17. The method as defined by claim 1 wherein determining that the variable has attained or passed through a threshold value comprises determining that the concentration of cancer cells in the CSF is at or below a threshold concentration value.

18. A method of reducing cancer cells in a patient’s cerebrospinal fluid (“CSF”), the method comprising:
forming a fluid circuit comprising fluidly coupling a first catheter with the patient’s brain ventricle and fluidly coupling a second catheter with the patient’s lumbar spine;
coupling the first and second catheters to a pump system;
using the pump system to increase the flow rate of the CSF through the CSF fluid circuit after forming the fluid circuit;
filtering cancer cells from the CSF through the fluid circuit when the flow rate is increased;
receiving feedback relating to cancer cell concentration in the CSF when filtering the cancer cells; and
controlling the pump to enable the CSF flow rate to return to a natural CSF flow rate in response to the received feedback.

19. The method as defined by claim 18 wherein a filter is in fluid communication with the fluid circuit, further wherein the filter comprises one or more of an electromechanical filter, a mechanical filter, a biochemical property filter, a physiochemical property filter, a temperature filter, and/or a bispecific affinity filter.

20. The method as defined by claim 18 wherein the feedback comprises the concentration of the cancer cells in the CSF.

21. The method as defined by claim 18 wherein the feedback is used to calculate a variable indicative of cancer cell concentration in the CSF.

22. The method as defined by claim 18 further comprising adding a therapeutic material to the CSF after filtering.

23. The method as defined by claim 18 further comprising dynamically varying the flow rate of the CSF through the CSF fluid circuit as a function of the feedback.

24. The method as defined by claim 18 wherein the fluid circuit comprises a filter cartridge through which the CSF flows.

25. A patient cerebrospinal fluid (“CSF”) management system comprising:
a plurality of catheters configured to cooperate to fluidly communicate the patient’s brain ventricle and the patient’s lumbar;
a filter in fluid communication with the plurality of catheters, the filter configured to filter or ameliorate cancer cells from the CSF through the plurality of catheters;
a spectroscopic instrument configured to produce an output signal having information relating to the CSF composition;
a pump; and
a flow controller operatively coupled with the pump and configured to cause the pump to control the flow rate of the CSF through the plurality of catheters, the flow controller configured to cause the pump to flow the CSF at a rate that is greater than the natural CSF flow rate of the patient,
the flow controller configured to cause the pump to permit restoral of the natural CSF flow rate in response to a determination that a variable indicative of cancer cell concentration in the CSF has attained or passed through a threshold value, the variable indicative of cancer cell concentration in the CSF being a function of the output of the spectroscopic instrument.

26. The system as defined by claim 25 wherein the spectroscopic instrument comprises one or more of nuclear magnetic resonance spectrometry, infrared spectrometry, ultraviolet visible spectrometry, flow cytometry and x-ray crystallography.

27. The system as defined by claim 25 wherein the flow controller is configured is configured to stop the pump to permit restoral of the natural CSF flow rate.

28. The system as defined by claim 25 wherein the filter comprises one or more of an electromechanical filter, a mechanical filter, a biochemical property filter, a physiochemical property filter, a temperature filter, and/or a bispecific affinity filter.

29. The system as defined by claim 26 wherein the variable comprises the concentration of the cancer cells in the CSF.

30. The system as defined by claim 26 wherein the flow controller is configured to calculate the variable from the spectroscopic instrument output signal.

31. The system as defined by claim 27 wherein the variable includes the concentration of a prescribed type of non-cancer cell in the CSF.

32. The system as defined by claim 26 wherein the flow controller is configured to dynamically vary the flow rate of the CSF as a function of the determined indication of cancer cell concentration in the CSF.

33. The system as defined by claim 26 wherein the filter comprises a cartridge through which the CSF flows.

34. A computer program product for use on a patient cerebrospinal fluid (“CSF”) management system for managing a closed loop CSF circuit between two points on a patient’s body having CSF with a natural flow rate, the computer program product comprising a tangible, non-transient computer usable medium having computer readable program code thereon, the computer readable program code comprising:
program code for controlling a pump having a pump outlet to direct CSF from the pump outlet at a CSF rate;
program code for controlling the CSF rate to be different than the natural rate to enable filtering of cancer cells;
program code for controlling the CSF rate to be equal to or less than natural rate when the concentration of cancer cells in the filtered CSF reaches a threshold value;
program code for adding the therapeutic material to the CSF via a therapeutic inlet into the CSF circuit, the therapeutic material being added to the CSF circuit at a therapeutic rate; and
program code for controlling the CSF rate to be different than the therapeutic rate.

35. The computer program product as defined by claim 34 wherein the CSF rate is a constant rate.

36. The computer program product as defined by claim 34 wherein the CSF rate varies over time.

37. The computer program product as defined by claim 34 wherein the CSF circuit is configured so that the CSF simultaneously flows at different rates at two different locations of the CSF circuit.

38. The computer program product as defined by claim 34 wherein the CSF circuit accesses one or more CSF-containing compartments within patient anatomy, including one or more of the lateral ventricles, the lumbar thecal sac, the third ventricle, the fourth ventricle, and the cisterna magna.

39. The computer program product as defined by claim 1 wherein the two points on the body comprise ports that permit access to the interior of the patient.

40. The computer program product as defined by claim 1 wherein the CSF circuit comprises an external catheter, the computer program product further comprising program code to track and limit use of the catheter.
